SEQUENCE LISTING (25 sequences)

SEQ ID NO: 1 - DNA, 40 bp, Artificial Sequence
  Description of Artificial Sequence Primer
catctcgagcggccgctttttttttttttttttttttttt

SEQ ID NO: 2 - DNA, 26 bp, Artificial Sequence
  Description of Artificial Sequence Primer
gggtttttggattaagttcttcattc

SEQ ID NO: 3 - DNA, 21 bp, Artificial Sequence
  Description of Artificial Sequence Primer
tcaccttccctccaaccagtt

SEQ ID NO: 4 - DNA, 30 bp, Artificial Sequence
  Description of Artificial Sequence Primer
caccctttgtgttcccaattccttccttag

SEQ ID NO: 5 - DNA, 21 bp, Artificial Sequence
  Description of Artificial Sequence Primer
cacactgcccctctcacacat

SEQ ID NO: 6 - DNA, 23 bp, Artificial Sequence
  Description of Artificial Sequence Primer
catttccctcgtttttctttgaa

SEQ ID NO: 7 - DNA, 22 bp, Artificial Sequence
  Description of Artificial Sequence Primer
ctccagttcgctgtccggccct

SEQ ID NO: 8 - DNA, 20 bp, Artificial Sequence
  Description of Artificial Sequence Primer
acactcagacccccaccaca

SEQ ID NO: 9 - DNA, 19 bp, Artificial Sequence
  Description of Artificial Sequence Primer
cataggcccctcccctctt

SEQ ID NO: 10 - DNA, 28 bp, Artificial Sequence
  Description of Artificial Sequence Primer
tctcccctcctcacagttgccatgtaga

SEQ ID NO: 11 - DNA, 23 bp, Artificial Sequence
  Description of Artificial Sequence Primer
cgaaatgctacaaaatgaagcaa

SEQ ID NO: 12 - DNA, 31 bp, Artificial Sequence
  Description of Artificial Sequence Primer
ttgtcttcatattttctttctaaatctttga

SEQ ID NO: 13 - DNA, 35 bp, Artificial Sequence
  Description of Artificial Sequence Primer
aactcttttagctcaagcaaatagcaagctgcaac

SEQ ID NO: 14 - DNA, 23 bp, Artificial Sequence
  Description of Artificial Sequence Primer
gaggagggattaaaagcacaaca

SEQ ID NO: 15 - DNA, 27 bp, Artificial Sequence
  Description of Artificial Sequence Primer
taagaacaaaatgatgagtgacagaca

SEQ ID NO: 16 - DNA, 31 bp, Artificial Sequence
  Description of Artificial Sequence Primer
ctcctgatcaacagcatcactgagcttcttt

SEQ ID NO: 17 - DNA, 26 bp, Artificial Sequence
  Description of Artificial Sequence Primer
ttttaaaagattcggctctgttattg

SEQ ID NO: 18 - DNA, 28 bp, Artificial Sequence
  Description of Artificial Sequence Primer
ttgaaaatgtagctgttataaggatggt

SEQ ID NO: 19 - DNA, 38 bp, Artificial Sequence
  Description of Artificial Sequence Primer
aatcaggctccgagaatccatgtatatatttgaactaa

SEQ ID NO: 20 - DNA, 18 bp, Artificial Sequence
  Description of Artificial Sequence Primer
aactcggcccccaacact

SEQ ID NO: 21 - DNA, 23 bp, Artificial Sequence
  Description of Artificial Sequence Primer
cctaggcccctcctgttattatg

SEQ ID NO: 22 - DNA, 28 bp, Artificial Sequence
  Description of Artificial Sequence Primer
catctccctcacaatttccatcccagac

SEQ ID NO: 23 - DNA, 20 bp, Artificial Sequence
  Description of Artificial Sequence Primer
ggctcggtgaccaaggtaaa

SEQ ID NO: 24 - DNA, 28 bp, Artificial Sequence
  Description of Artificial Sequence Primer
tccatacaaaagtaggtggttaaaaaca

SEQ ID NO: 25 - DNA, 31 bp, Artificial Sequence
  Description of Artificial Sequence Primer
accgaggcagtcatctacaaataacccatca

(19) United States
(12) Patent Application Publication
(10) Pub. No.: US 2004/0241838 A1
(43) Pub. Date: Dec. 2, 2004

(54) Stem cells

(76) Inventors: Penelope Ann Johnson, Glossop (GB); Richard Gregory Wolowacz, Glossop (GB)

Correspondence Address: HELLER EHRMAN WHITE & MCAULIFFE LLP, 1666 K STREET,NW, SUITE 300, WASHINGTON, DC, 20006, US

(21) Appl. No.: 10/479,342

(22) PCT Filed: May 31, 2002
(86) PCT No.: PCT/GB02/02691

(30) Foreign Application Priority Data
May 31, 2001 (GB) 0113118.4

(51) Int. Cl. C12N 5/08; C12N 15/85

(52) U.S. Cl. 435/366; 435/455

(57) ABSTRACT

The present invention relates to methods of producing pluripotential mammalian stem cells by reprogramming somatic cells, as well as stem cells obtained by the methods, and uses of these stem cells. In one aspect, a method of producing a stem cell from a target mammalian somatic cell involves introducing into the target cell a medium which includes or consists of a whole, partial or derivative extract of a reprogramming cell, wherein the extract comprises soluble components of cytoplasm and nuclear factors and wherein the extract is enriched for the nuclear factors.

The present invention relates to stem cells. More particularly, it relates to methods of producing pluripotential mammalian stem cells by reprogramming somatic cells, as well as stem cells obtained by the methods, and uses of these stem cells.

The first examples of nuclear reprogramming were provided by Briggs and King (Briggs & King, 1952, Proc. Natl Acad. Sci.38: 455-463; King & Briggs, 1956, Cold Spring Harb. Symp. Quant. Biol. 21: 271-289) and Gurdon and coworkers (Gurdon et al., 1975, J. Embryol. Exp. Morphol. 34: 93-112; Gurdon et al., 1979, Int. Rev. Cytol. Suppl. 9:161-178). Nuclei derived from the differentiated somatic cells of amphibia transplanted into enucleated eggs of frogs were demonstrated to retain the capacity to direct development of tadpoles. Later experiments extended these observation with the development from transplanted nuclei of adult frogs (Gurdon et al., 1975, supra). In the 1990's further experiments demonstrated that sheep could develop from nuclei of somatic ovine cells transplanted into enucleated ovine oocytes (Campbell et al., 1996, Nature 380: 64-66).

It has since been shown that somatic cell nuclei can be reprogrammed by injecting them into enucleated oocytes to produce stem cells (WO 97/07668 and WO 97/07669).

Reprogramming can occur if germ cells or teratocarcinoma cells are fused with somatic cells, for example with thymocytes, to produce stem cells (WO 00/49137 and WO 00/49138).

In isolated quiescent nuclei of Xenopus laevis, erythrocytes can be reactivated using Xenopus egg extract to provide isolated replicative nuclei (Wangh et al., 1995, J. Cell Sci. 108: 2187-2196). These nuclei did not constitute cells as such, as they had no cytoplasm bounded by an outer cell membrane: such experiments provide an example of a cell-free model or surrogate of nuclear reactivation. Similarly, Kikyo et al. (2000, Science 289:2360-2362) demonstrated in a cell-free model system the morphological remodelling of somatic nuclei incubated in soluble extracts made from Xenopus laevis eggs. Remodelling in this cell-free surrogate system was accompanied by loss of specific proteins from the chromatin of the nucleus and gain of other factors by ingress to the nucleus from the Xenopus egg extract.

However, although exchange of specific protein factors between Xenopus nuclei and Xenopus egg extracts has been observed in such cell-free surrogate model systems, and similar exchange of proteins is implied in experiments in which mammalian somatic nuclei have been introduced into enucleated mammalian oocytes to produce cloned animals, similar observations have not been noted in isolated somatic mammalian nuclei treated with extract preparations either from Xenopus eggs/oocytes or EC cells.

In some cases there may be practical problems associated with cell manipulation, especially cell reprogramming techniques. These include the size of the cells and nuclei used in the techniques and the ability of the skilled person to manipulate them. Thus, while it is possible to inject a somatic nucleus into an enucleated oocyte, the successful implantation of a somatic nucleus within an enucleated somatic cell by the same techniques has not been reported.

Moreover, there are other issues associated with the use of oocyte based reprogramming to provide stem cells, such as the availability and ethical acceptability of using human oocytes or embryos as the sources of cells.

Adult stem cells are rare. Only an estimated 1 in 10 000 to 15 000 cells in the bone marrow is a haemapoietic stem cell (NIH “Stem Cells: Scientific Progress and Future Research Directions” [Kirschstein & Skirboll, June 2001]). This has significant process costs implications, and by necessity quite large samples would be required, for isolation of useful quantities of adult stem cells. Furthermore, stem cells would need to be purified from the sampled tissue to develop therapies. In the prior art there are reported methods of purifying stem cells from bone marrow and skin.

According to the present invention there is provided in a first aspect a method of producing a pluripotential mammalian stem cell (“stem cell”) from a target mammalian somatic cell (“target cell”) comprising the steps of:

(i) providing a medium which includes or consists of a whole, partial or derivative extract of a reprogramming cell, wherein the extract comprises soluble components of cytoplasm and nuclear factors, and wherein the extract is enriched for the nuclear factors;

(ii) providing a target cell comprising a nucleus and an outer cellular membrane;

(iii) introducing the medium into the target cell, wherein the medium causes reprogramming of the target cell nucleus to form a stem cell having a reprogrammed nucleus and an outer cell membrane from the target cell.

The invention provides an effective method for the generating stem cells from somatic target cells. The stem cells can be produced without the need to isolate stem cells from patients.

The reprogramming cell is preferably not obtained from a human embryo or a human oocyte.

The term “stem cell” as used herein refers broadly to a cell which is pluripotential, i.e. a cell which has the capacity to give rise to two or more tissues or a type of tissue which is distinct from the originating cell. This widely used meaning of stem cell thus encompasses stricter definitions of both stem cells and progenitor or precursor cells. For example, stem cell as used herein encompasses the strict definition of stem cell and progenitor or precursor cell outlined in the NIH Stem Cells report (supra). The report defines a stem cell as “a cell from the embryo, fetus, or adult that has the ability to reproduce itself for long periods or, in the case of adult stem cells, throughout the life of the organism. It can also give rise to specialized cells that make up the tissues and organs of the body. Much basic understanding about embryonic stem cells has come from animal research. In the laboratory, this type of cell can proliferate indefinitely, a property that is not shared by adult stem cells”. The report defines “progenitor or precursor cell” as a cell which “occurs in fetal or adult tissues and is partially specialized; it divides and gives rise to differentiated cells. Researchers often distinguish precursor/progenitor cells in the following way: when a stem cell divides, one of the two new cells is often a stem cell capable of replicating itself again. In contrast, when a progenitor/precursor cell divides, it can form more progenitor/precursor cells or it can form two specialized cells, neither of which is capable of replicating itself. Progenitor/precursor cells can replace cells that are damaged or dead, thus maintaining the integrity of a tissue such as liver or brain.”

The term “pluripotential” is regarded as synonymous with “pluripotent”. As used herein, pluripotential covers a stem cell which is not committed to differentiate only towards one given adult phenotype. This commonly used meaning, which could be referred to as “multipotent”, is to be distinguished from a strict definition of “pluripotent stem cell“given in the above NIH report as a “single pluripotent stem cell has the ability to give rise to types of cells that develop from the three germ layers (mesoderm, endoderm and ectoderm) from which all the cells in the body arise. The only known sources of human pluripotent stem cells are those isolated and cultured from fetal tissue that was destined to be part of the gonads”. Here, the term pluripotential encompasses stem cells which give rise to different lineages within the same germ layer.

A somatic cell is defined herein as a diploid cell of any tissue/structural type that does not contribute to the propagation of the genome beyond the current generation of the organism. All cells save germ cells are somatic cells and give rise to the individual body.

Nuclear factors refers to proteins (or RNAs) normally bound within the nuclear membrane (except during mitosis in somatic cells and meiosis in germ cells). Nuclear factors may include heteronuclear RNA (“hnRNA”, i.e. messenger RNA prior to processing and export). The hnRNA may encode reprogramming factors. The nuclear factors may include DNA binding proteins bound in chromatin to the chromosomes, for example histones, transcription factors and other ancillary factors that may affect gene expression (either directly or indirectly).

Reprogramming is defined as a process by which a specific functional phenotype of a differentiated cell is expunged to yield a cell with a different functional phenotype. Deprogramming is a type of reprogramming in which an original specific functional phenotype in a differentiated cell is expunged to yield a cell without a differentiated phenotype, to render that cell undifferentiated or more pluripotent, for example, a thymocyte could be deprogrammed to resemble an embryonic stem cell. Reprogramming and deprogramming can be used interchangeably since to remove the differentiated function from a cell to yield a more pluripotent stem-like cell is equivalent to reprogramming that cell to perform a new function, that being the function of a pluripotent, stem-like cell. Also, a cell once deprogrammed could be reprogrammed to express a new function, for example, a fibroblast could be deprogrammed to yield a pluripotent cell then reprogrammed to express thymocyte functions.

Preferably, the soluble components and/or the nuclear factors may cause reprogramming of the target cell nucleus in step (iii).

One advantage of the present invention is that the effectiveness of the extract prepared from the reprogramming somatic cells is improved by the presence of soluble components and nuclear factors. Clearly, the soluble components and nuclear factors will need to be of a sufficient concentration in the extract to be cause programming.

The extract may be from a reprogramming cell in a G1, G2 or M cell cycle phase or in a metaphase to anaphase transition cell cycle phase. Extracts made from cells at a specific phase of the cell life-cycle can contain factors effective in deprogramming that are preferentially present and active only during that particular phase. Cells collected from a single cell cycle phase can be expected to yield the maximal concentration of the particular factors present only within that phase. During M-phase (mitosis or meiosis) the nuclear envelope is broken down and nuclear and cytoplasm components are found within the same soluble cytosol at physiological concentrations and stoichiometry.

The cell cycle phase of the reprogramming cell may be induced by a synchronisation agent. The synchronisation agent may for example be Nocodazole.

The nuclear factors may be obtained from a karyoplast isolated from the reprogramming cell. Alternatively, the nuclear factors may be obtained from a nucleus isolated from the karyoplast or the reprogramming cell.

The nuclear membrane of the reprogramming cell, of the karyoplast or of the isolated nucleus may disrupted to release nuclear factors. The nuclear membrane may be disrupted by sonication, by isotonic bursting, and/or by using an homogeniser.

The medium may be introduced into the target cell following permeabilisation of the outer cellular membrane of the target cell.

Permeabilisation may be achieved using a permeabilisation agent, for example saponin, digitonin or streptolysin O. Saponin may be used at 5-45 μg/ml, preferably at 10-35 μg/ml, for example at 30 μg/ml. Streptolysin 0 may be used at 1-20 units/ml, for example at 5-10 units/mi.

Permeabilisation may be achieved using an electric pulse.

The permeabilisation of the outer cell membrane of the somatic cell can cause leaching of cytosol from the somatic cell into the surrounding milieu. The extract of the reprogramming cell maybe added to the milieu containing the permeabilised cell. The soluble components and/or nuclear factors of the extract may permeate into the somatic cell and thereby causing reprogramming of the nucleus to form a stem cell.

The medium may be injected into the target cell.

The extract may be from a reprogramming cell which has been pre-treated with an agent that causes enucleation of the cell. For example the agent may be cytochalasin, preferably cytochalasin B or D, for example at 1-20 μM. Such agents inhibit intermediate filament production and stabilisation, thereby aiding release of the mitotic/meiotic spindle or nucleus from the cell.

The extract may be provided as enucleated whole cytoplasm. Alteratively, the extract may be provided as a derivative of the cytoplasm of the reprogramming cell. In a further embodiment, the extract is provided as a derivative of an isolated karyoplast.

The extract and/or medium may be supplemented with a ribonuclease inhibitor and/or a proteinase inhibitor. This will prevent or minimise degradation of RNAs and/or proteins by cellular ribonucleases and/or proteases.

The extract and/or medium may be supplemented with an antioxidant, for example dithiothreitol (preferably at 0.5-5 mM) and/or β-mercaptoethanol (preferably at 100-500 mM), to prevent or minimise inactivation of reprogramming factors through oxidation.

The extract and/or medium may be supplemented with an agent which inhibits protein dephosphorylation, for example β-glycerophosphate and/or vanadate.

Addition of such an agent prevents or minimises protein dephosphorylation from inactivating reprogramming factors.

The extract and/or medium may be supplemented with an energy regeneration system/mix comprising creatine kinase (for example at 50-100 μg/ml) and/or creatine phosphate (for example at 10-20 mM) and/or ATP (for example at 1-2 mM) and/or GTP (for example at 1-2 mM) and/or MgCl₂ (for example at 1 mM). The energy regeneration mix supplements biochemical energy in vitro.

The extract and/or medium may be supplemented with an agent that stabilises the extract and/or medium, for example glycerol and/or sucrose (preferably at 5-50% v/v). Stabilisation may be during preparation of the extract and/or medium or during storage.

The method of the invention may comprise a further step of:

(iv) incubating the target cell in conditions conducive to the reconstruction and/or repair of the outer cellular membrane to form the stem cell.

The reprogramming cell may be a germ cell, for example an egg cell, or an embryonal carcinoma (EC) cell. A germ cell is defined therein as a haploid cell capable of propagating the genome into the next generation. Germ cells are distinguished by their reproductive function/capacity. Such cells may develop into oocytes in a female. Oocytes in turn may mature into eggs. A haploid cell contains one copy of each chromosome while a diploid cell contains two copies of each non-sex-determining chromosome and a full complement of sex-determining chromosomes particular to the species. EC cells are defined therein as pluripotent cells believed to be the stem cells (equivalent to ES cells but not embryonally-derived) that give rise to all other cell types in teratomas or germ cell tumours except seminoma (which are probably the primordial germ cells from which the tumours arise). The EC cells are preferably disrupted by homogenization in a Dounce homogeniser or by sonication, and the separation of the cellular components made by centrifugation.

Alternatively, the germ cell may be aXenopus laevis egg cell or oocyte. Disruption of the eggs/oocytes and subsequent separation of the cellular components are preferably made by centrifugation according to well-known methods.

The reprogramming cell may be a mammalian cell.

The target cell may be a thymocyte, peripheral blood lymphocyte, epidermal cell, buccal cavity cell, cumulus cell, bone marrow stem cell, nervous system stem cell or gut stem cell, or is obtained from established cell lines, tissues or organs of an adult mammal.

The target cell nucleus may be encapsulated in a support medium, for example agarose.

The method of the invention may further comprise the step of:

(v) isolating at least one stem cell.

The method of the invention may yet further comprise the step of:

(vi) culturing the stem cell produced by the method in conditions conducive to propagate the stem cell.

In a further aspect, there is provided a method for the simultaneous production of stem cells, comprising incubating more than one target cell in the medium as defined above so as to induce simultaneous reprogramming of target cell nuclei. This method does not require the manipulation of individual reprogramming cells and nuclei. This method has an advantage in that a large number of somatic cell nuclei may be reprogrammed at the same time.

In addition, the reprogramming method of the invention may be used on more than one target cell type at a time. Therefore there may be no absolute requirement for purification/selection of different potential target cells from the mixture of cells explanted from a patient sample.

Further provided according to the present invention is a stem cell obtained or obtainable by the methods described above.

In one embodiment, the stem cell according to the invention has the ability to proliferate in culture in an undifferentiated state.

The stem cell may have at least one pluripotential characteristic. The stem cell may have the ability to differentiate into one of at least two selected tissue types.

The stem cell may expresses at least one selected marker. The selected marker may be one or more of the following: Oct3/4, Sox2, SSEA-1 (−), SSEA-3 (+), SSEA-4 (+), TRA-1-60 (+), TRA-1-81 (+), lacZ and GFP.

The stem cell may possess telomerase activity.

The stem cell may possess a chromosomal methylation pattern characteristic of pluripotential cells.

The stem cell may be human.

In a further aspect of the invention there is provided a cell culture comprising at least one stem cell produced according to any one of the above methods. The cell culture may comprise at least one stem cell as defined above.

Further provided according to the present invention is the use of a stem cell produced according to any one of the above methods in the production of one or more of the following tissues: neural, smooth muscle, striated muscle, cardiac muscle, bone, cartilage, liver, kidney, respiratory epithelium, haematopoietic cells, spleen, skin, stomach, and intestine.

The invention includes therapies using target cells from both autologous and allogeneic sources. In a preferred embodiment, a population of what are effectively pluripotent/multipotent “autologous adult stem cells” is produced.

Autologous approaches have two theoretical advantages over allogeneic approaches in terms of (a) requirements for immunosuppression/tolerance and (b) of transmission of infective agents. However as there is no transfer of nuclear DNA in the invention, the reprogrammed target cells should retain the target cell MHC, and would be immunologically autologous and this addresses point (a). Preferably the reprogramming extracts should be derived from ethically obtained and virally screened master cell banks (validated by regulatory authorities) of somatic cells (for example embryonal carcinoma cells) to address point (b).

Yet further provided is a tissue for use in transplantation comprising at least one stem cell of the invention.

Also provided is a therapeutic composition comprising at least one stem cell of the invention. The therapeutic composition may comprise a suitable excipient, diluent or carrier. The therapeutic composition may be used in tissue transplantation.

In a further aspect of the invention there is provided a method for inducing differentiation of at least one stem cell comprising the steps of:

(i) providing a stem cell according to the invention;

(ii) culturing the stem cell under conditions which cause differentiation of the stem cell; and optionally

(iii) storing the differentiated stem cell prior to use under suitable storage conditions.

In yet a further aspect of the invention there is provided a method of producing a tissue comprising the steps of:

(i) providing a stem cell produced according to the invention; and

(ii) culturing the stem cell under conditions which causes proliferation of the cell to form a tissue.

Further provided is a method to treat a condition or a disease requiring transplantation of a tissue comprising the steps of:

(i) providing a tissue produced according to the invention;

(ii) transplanting the tissue into a patient to be treated; and

(iii) treating the patient under conditions which allows the acceptance of the transplanted tissue by the patient.

Also provided is a therapeutic composition comprising a tissue produced according the invention. The therapeutic composition may further comprise a suitable excipient, diluent or carrier.

In another aspect there is provided the use of a stem cell produced according to the invention for screening of compounds with potential to treat disease.

In a further aspect there is provided the use of a differentiated stem cell produced according to the invention or a tissue produced according to the invention, for screening of compounds with potential to treat disease.

Also provided is the use of a stem cell produced according to the invention or a differentiated cell produced according to the invention, in a study of organ development.

In another aspect there is provided a reprogrammed mammalian cell produced by transfer of an extract that includes or consists of soluble components of whole, part or derivative of another type of cell, wherein the extract is enriched for nuclear factors.

In yet a further aspect there is provided a medium or extract obtained from a mammalian reprogramming cell, wherein the medium or extract is as defined herein.

The invention may in a further aspect be stated as a method of producing a pluripotential mammalian stem cell from a target mammalian somatic cell comprising the steps of:

(1) providing a medium which includes or consists of an extract of a reprogramming cell (for example, a mammalian reprogramming cell), which extract comprises soluble components of cytoplasm from said cell;

(2) providing a target mammalian somatic cell having at least a nucleus and an outer cellular membrane;

(3) permeabilising said outer membrane;

(4) incubating the somatic cell in the medium and allowing said soluble components of cytoplasm to permeate from the medium into the cell wherein said components cause reprogramming of the somatic cell nucleus to form a stem cell having a reprogrammed nucleus and an outer cell membrane from the target somatic cell; and optionally a further step of:

(5) incubating the reprogrammed cell in conditions conducive to the reconstruction and/or repair of the permeabilised outer cellular membrane to form the stem cell.

Alternatively, in step (3) above said extract may be injected into the somatic cell.

General aspects of methodologies suitable for the invention are summarised below.

Incubation of cell with cytochalasins, such as cytochalasin B, will induce cells to exclude the nucleus. Incubation of the cell may then be followed by centrifugation through a density gradient, typically composed of Ficoll. In the case of eggs, oocytes and EC cells treated with Nocodazole, the nucleus ceases to exist as a discrete organelle, the nuclear membrane having been disassembled in the process of entering meiosis (oocytes, eggs) or mitosis (EC cells treated with Nocodazole).

Alternatively, enucleation of cells to yield both cytoplasts (enucleated cells) and karyoplasts (extruded nuclei which retain a thin rim of cytoplasm and are surrounded by a plasma membrane) may be achieved by well-established techniques in which the cells, growing attached to a plastic disc, are inverted over a solution of cytochalasin B in a centrifuge tube and centrifuged. The cytoplasts remain attached to the plastic disc, while the karyoplasts are pelleted at the bottom of the centrifuge tube. An alternative well-established method is to separate the karyoplasts from the cytoplasts by centrifugation of cytochalasin-treated cells through a gradient composed of Ficoll. After appropriate centrifugation, the denser components such as karyoplasts pellet at the bottom of the tube whilst the lighter components such as the cytoplasts remain suspended in the gradient.

The separation of karyoplasts and cytoplasts enables the isolation of two separate cytosols made from the cytoplasm and the nuclei of the disrupted cells, either or both of which may then be used in deprogramming.

A large range of somatic cells derived from any tissue or organ of an adult mammal, particularly of human origin, may be used as the source of a nucleus as a target for deprogramming. Particular preferred somatic cell types include, but are not limited to, thymocytes, peripheral blood lymphocytes, epidermal cells such as from the buccal cavity, cumulus cells, or other stem cells isolated from biopsies of various tissues, such as the bone marrow, the nervous system and the gut. The technique may also be applied to various established cell lines such as those derived from the various tumours including, for example but not limited to, lymphoblatoid cell lines. Preferably, the target cell is easily obtainable using non-invasive methods, for example blood samples, buccal epithelial scrapes and/or skin biopsies.

The outer cellular membrane of the target somatic cell may be permeabilised through the use of specific agents (such as saponin, digitonin, streptolysin O). The cytosol of the cell after permeabilisation leaches into the surrounding milieu. Since these permeabilisation agents act principally on the outer cellular membrane, this treatment leaves the nucleus and nuclear membrane intact (known as a nucleoplast).

Alternatively, the outer cellular membrane of the target cell may be permeabilised by means of an electric pulse, and the cytosol allowed to leach into the surrounding milieu.

Once the cellular membrane has been permeabilised and the cytosol leached out, the nucleus may require support. If so, it may be encapsulated in a support medium, such as agarose.

The permeabilised somatic cells, leaving the nuclei, whether or not surrounded by support medium, may be combined with the extract from EC cells or from germ cells (for example, Xenopus eggs or oocytes) by incubating the two components together. The incubated permeabilised somatic cell, incubated with the extract to form a reprogrammed cell, may then be reintroduced into tissue culture conditions conducive to cellular reconstitution, reconstruction and/or repair.

To produce reprogrammed somatic cells so that they have pluripotential characteristics, the permeabilised somatic cells from mammalian tissues, cell lines or primary cells should be incubated in an amount of the extract sufficient to generate pluripotent cells.

Further, the combined cell requires appropriate conditions for the reprogramming of the differentiated cell nucleus.

To enhance the efficiency of the reprogramming, the differentiated somatic cell nucleus may be cultured in the presence of drugs that inhibit methylation or promote demethylation, for example 5-azacytidine. Drugs may also be used to alter the structure of chromatin, for example butyrate, spermine, trichostatin A or trapoxin which inhibit deacetylation and promote acetylation of histones, which play a role in X chromosome inactivation, gene imprinting and regulation of gene expression.

Many somatic cell nuclei may be reprogrammed at once without the need to separate and incubate the individual components. The outer cellular membrane of a number of target somatic cells may be permeabilised simultaneously. To these permeabilised cells sufficient quantity of extract of the reprogramming cell can be added to cause reprogramming of the nuclei. The mixture is then incubated under suitable conditions causing the reprogramming of the nuclei to form many stem cells.

The stem cells may be reconstituted, reconstructed and/or repaired by culturing the mixture in appropriate conditions. Alternatively, the stem cells may be isolated before being cultured in appropriate conditions.

This method would be used to produce a large number of stem cells without the need for individual manipulation of the components, which may be both time-consuming and difficult to perform on a routine basis.

Alternatively, the extract of the reprogramming cell, be it whole, partial or derivative, may be injected into the target somatic cell in sufficient quantity to cause the somatic cell nuclei to be reprogrammed to form a pluripotent cell.

The somatic cells to be reprogrammed may be genetically labelled. This enables easier identification of the reprogrammed cells. Genetic labels such as lac Z or GFP could be used.

Further, the target somatic cells may be genetically altered, such that if they are reprogrammed they would express a gene characteristic of a dedifferentiated cell. Oct 4 is one such suitable marker.

In all these studies, the cells may cultured in standard cell culture medium that include Dulbecco's modified Eagle's Medium (DME, high glucose formulation) or Ham's F12, supplemented in some cases with foetal bovine serum or with other additives. Subsequent to combining and reprogramming, the resultant reprogrammed stem cells may be grown on feeder layers of cells that include but are not limited to irradiated or mitomycin C treated STO cells, or embryonic fibroblasts of various species, including human.

In addition, if required the stem cells may be cultured in the presence of various growth factors or other tissue culture additives that include, but are not limited to, LIF, FGF, and SCF.

The stem cells produced by the methods described above have pluripotent properties that closely resemble those of embryonic stem cells, so that the cells are able to differentiate and initiate differentiation pathways that result in the formation of any cell type that may be found in the adult, embryo or in extra-embryonic tissues, given appropriate conditions. The maintenance of an embryonic stem cell state may be monitored by assay of various markers that included the cell surface antigens SSEA3, SSEA4, TRA-1-60, TRA-1-81, by their expression of alkaline phosphatase or by expression of Oct 3 or 4 (as above).

The stem cells should retain their stem cell phenotype when cultured on appropriate feeder cells. However, the cells may be differentiated under a variety of conditions.

The removal of the feeder layer of cells or culturing the cells in suspension, followed by replating the cells in the absence of the feeder cells in appropriate tissue culture flasks will result in the differentiation of the stem cells into a variety of cell and tissue types that include neural, smooth muscle, striated muscle, cardiac muscle, bone, cartilage, liver, kidney, respiratory epithelium, haematopopietic cells, spleen, skin, stomach, and intestine.

Differentiation of pluripotential cells may also be initiated by altered conditions affecting cell density and aggregation (for example seeding at low cell densities or trysinisation) or by forcing growth as a suspension (rather than adherent) culture by exposure to various agents that include, but are not limited to, retinoic acid (RA), and other retinoids, hexamethylene bisacetamide (HMBA), and the bone morphogenetic proteins (BMPs).

The type of cell that arises depends upon the nature of the inducing agent, and the culture conditions including the presence or absence of specific growth factors or other molecules.

The pluripotent stem cells have a number of uses.

The cells may be cultured such they differentiate into a selected cell type. This differentiated cell may then be used for drug screening.

Alternatively, the pluripotent cells may be used in basic cellular research. For example, in the study of cell-cell interactions in organ development.

Most usefully, the pluripotential cells may be used to produce selected differentiated cells which may be cultured and used in tissue transplantation and the treatment of disease.

The invention is further described in the experimental section below with reference to the accompanying figures, in which:

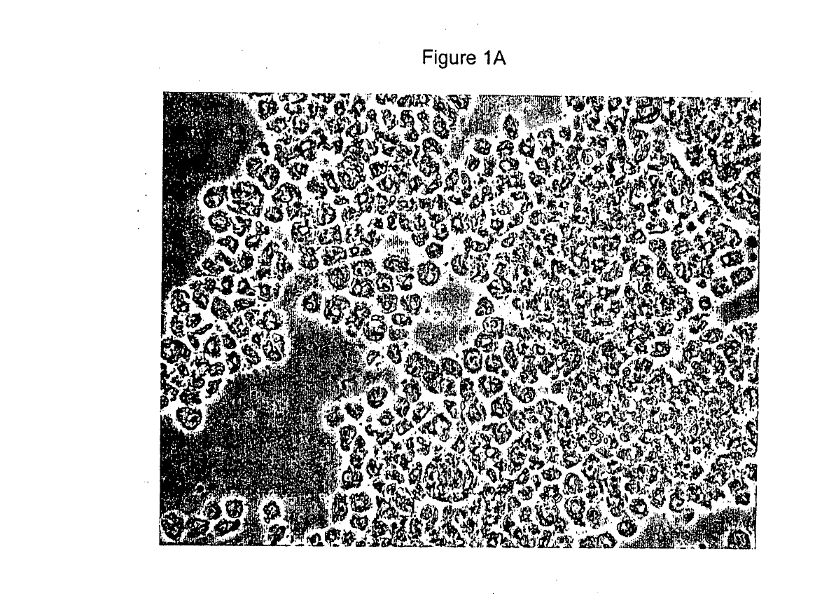

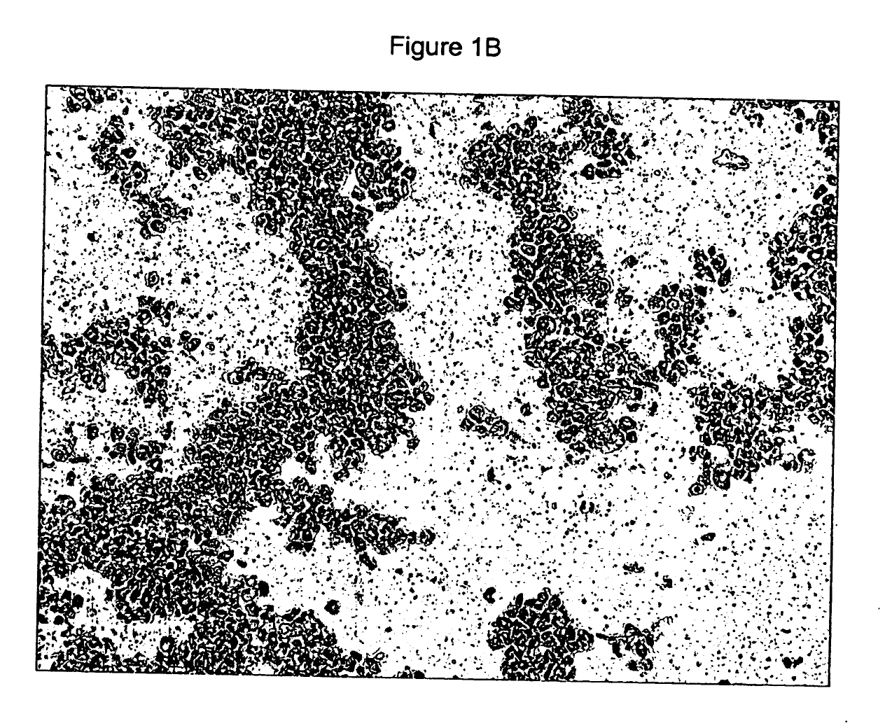

FIG. 1A Shows a micrograph of asynchronous human Embryonal Carcinoma (EC) cell line 2102E cells prior to homogenisation;

FIG. 1B Shows a micrograph of asynchronous 2102E cells after 20× Dounce homogenisation;

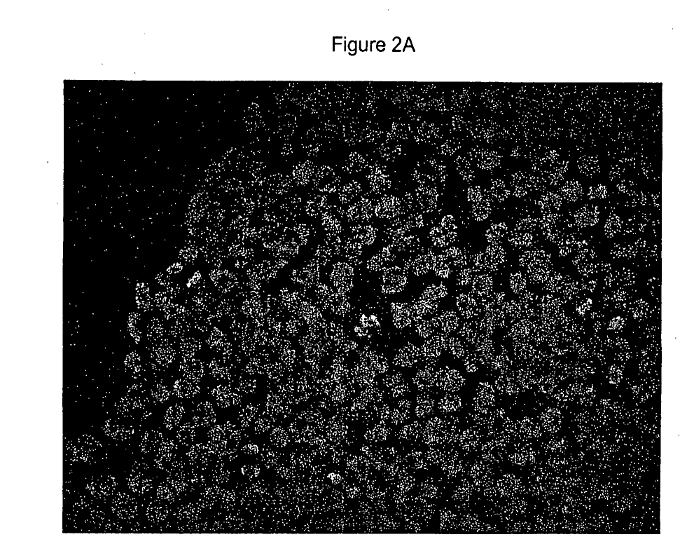

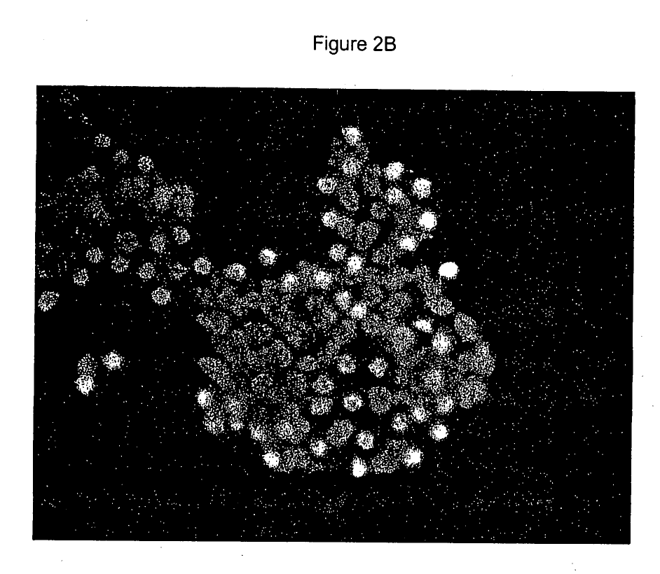

FIG. 2A Shows a micrograph of 2102E cells in the absence of Nocodazole treatment (UV illumination);

FIG. 2B Shows a micrograph of 2102E cells treated with 10 ng/ml Nocodazole (UV illumination);

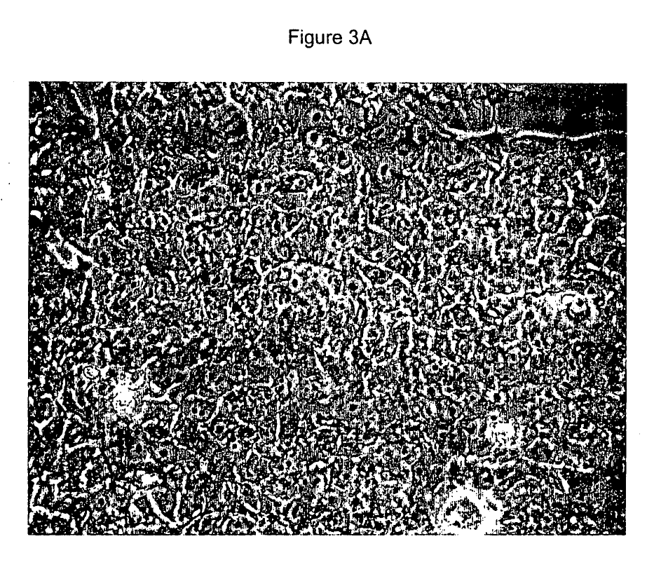

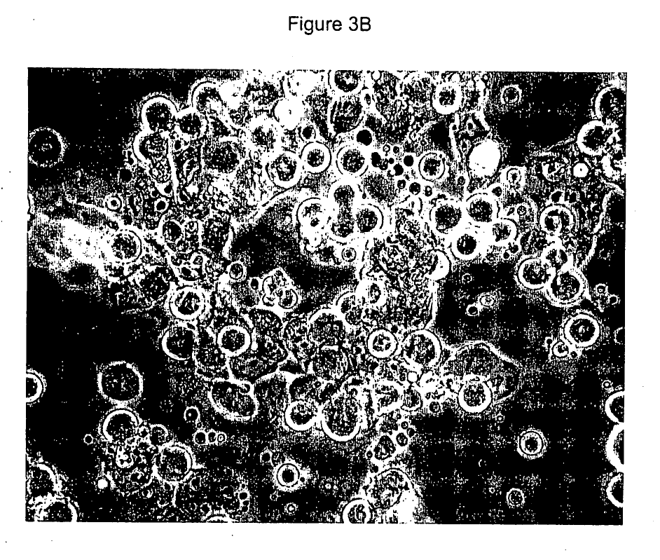

FIG. 3A Shows a micrograph of 2102E cells in the absence of Nocodazole treatment (transmitted light illumination);

FIG. 3B Shows a micrograph of 2102E cells treated with 10 ng/ml Nocodazole (transmitted light illumination);

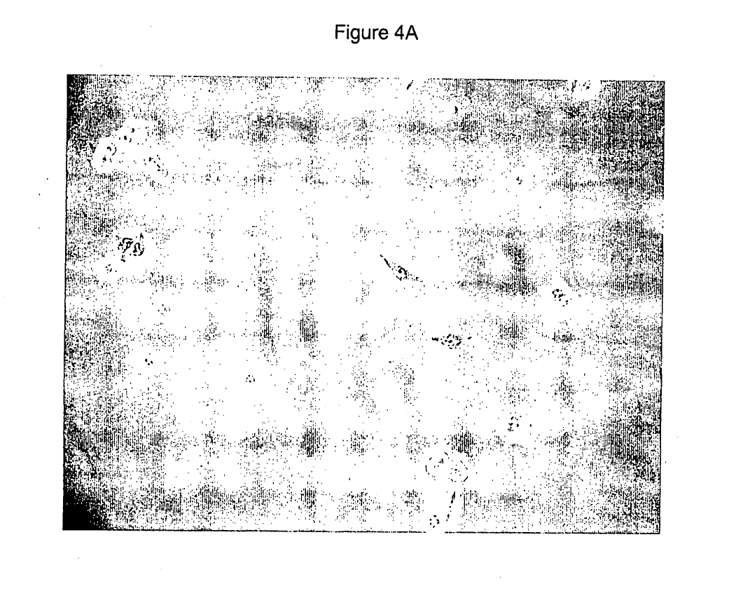

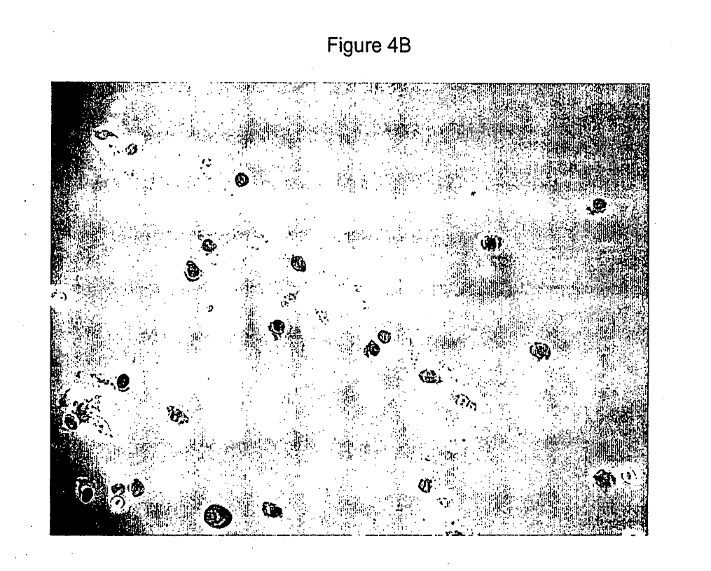

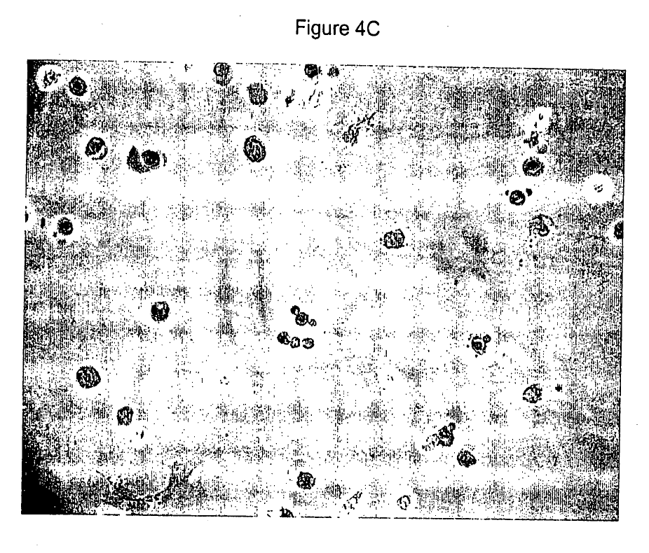

FIG. 4A Shows a micrograph of Chinese Hamster Ovary (CHO) EM9 cells harvested immediately after treatment with physiological buffer and stained with Trypan Blue;

FIG. 4B Shows a micrograph of CHO EM9 cells harvested immediately after 30 μg/ml saponin treatment and stained with Trypan Blue;

FIG. 4C Shows a micrograph of CHO EM9 cells harvested immediately after 50 μg/ml saponin treatment and stained with Trypan Blue;

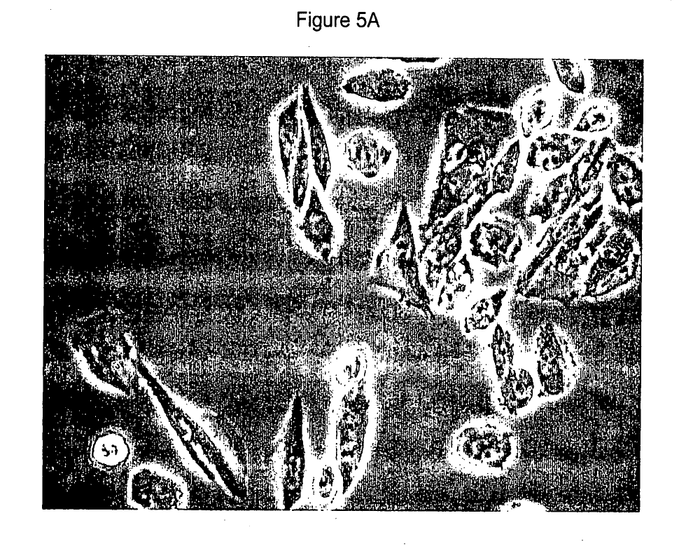

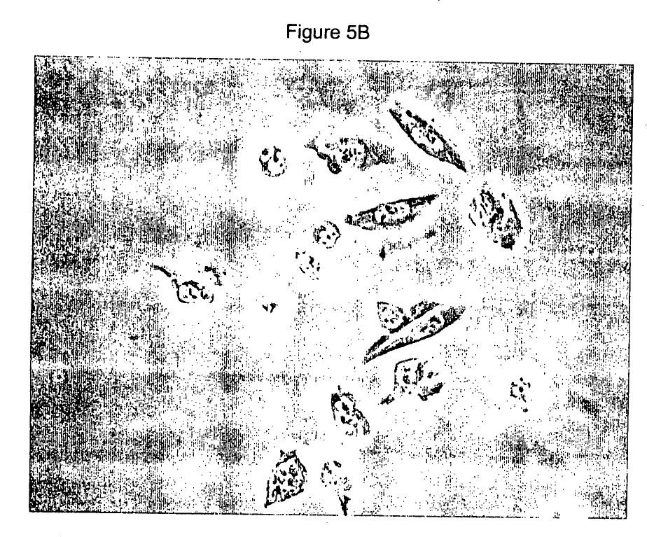

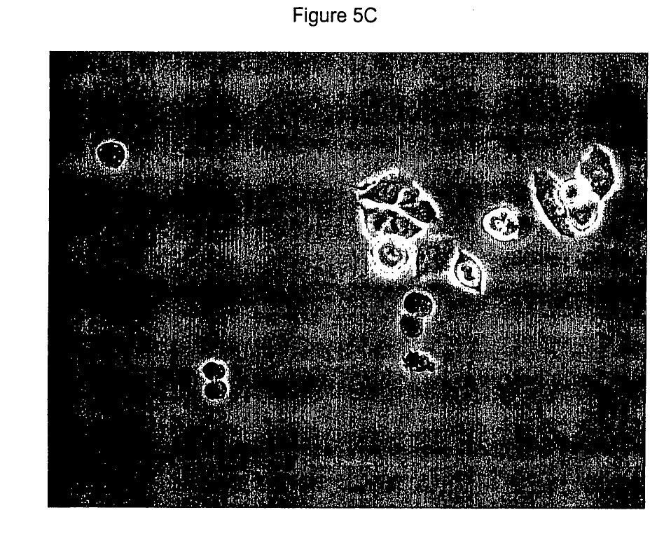

FIG. 5A Shows a micrograph of Chinese CHO EM9 cells harvested 24 h after treatment with physiological buffer and stained with Trypan Blue;

FIG. 5B Shows a micrograph of CHO EM9 cells harvested 24 h after 30 μg/ml saponin treatment and stained with Trypan Blue;

FIG. 5C Shows a micrograph of CHO EM9 cells harvested 24 h after 50 μg/ml saponin treatment and stained with Trypan Blue;

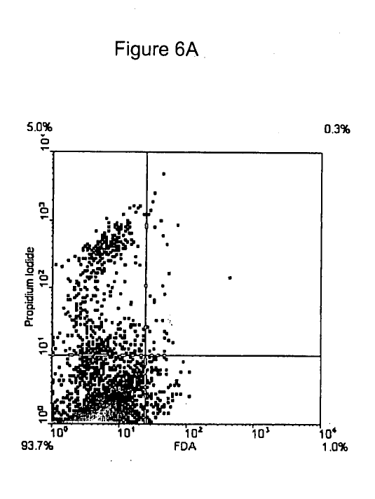

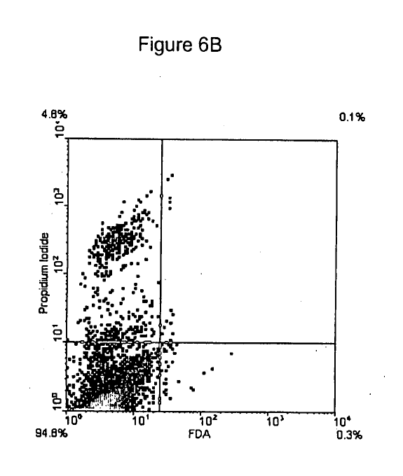

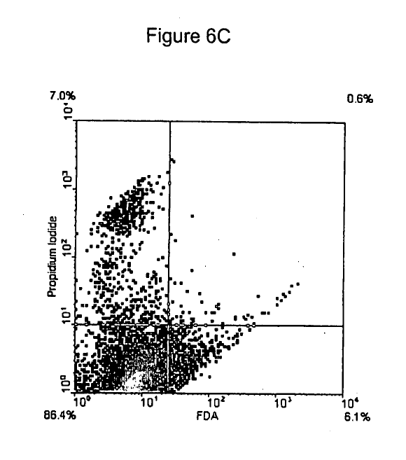

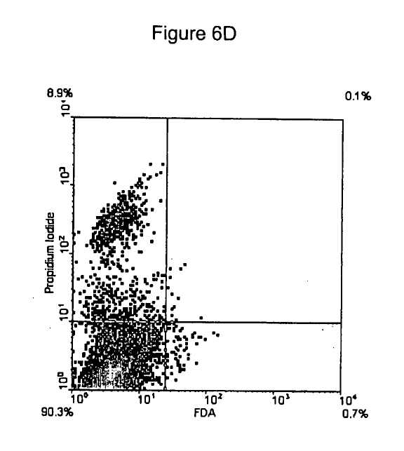

FIG. 6A Illustrates a flow cytometric analysis of CHO EM9 cells treated in the absence of streptolysin O and stained with fluorescein diacetate (FDA) and propidium iodide (PI);

FIG. 6B Illustrates a flow cytometric analysis of CHO EM9 cells treated with 5 units of streptolysin O and stained with FDA and PI;

FIG. 6C Illustrates a flow cytometric analysis of CHO EM9 cells treated with 10 units of streptolysin O and stained with FDA and PI;

FIG. 6D Illustrates a flow cytometric analysis of CHO EM9 cells treated with 20 units of streptolysin O and stained with FDA and PI;

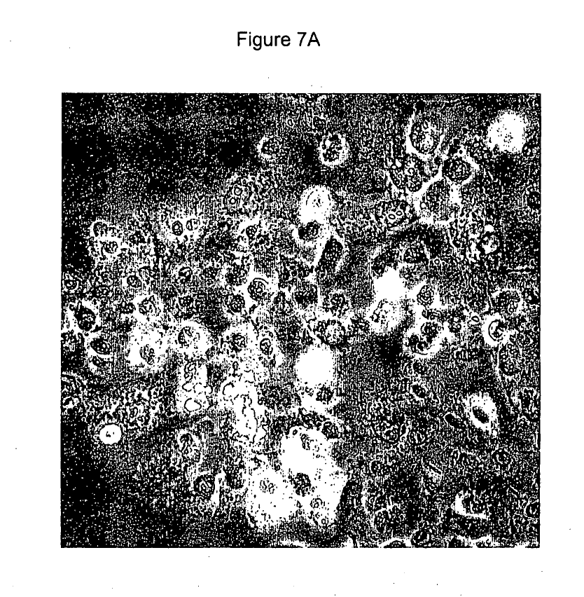

FIG. 7A Shows a micrograph of human osteosarcoma cell line 143B cells injected with extract containing fluorescein isothiocyanate (FITC)-conjugated-dextran (UV and transmitted white light illumination);

FIG. 7B Shows a micrograph of 143B cells injected with extract containing FITC-conjugated-dextran (UV illumination);

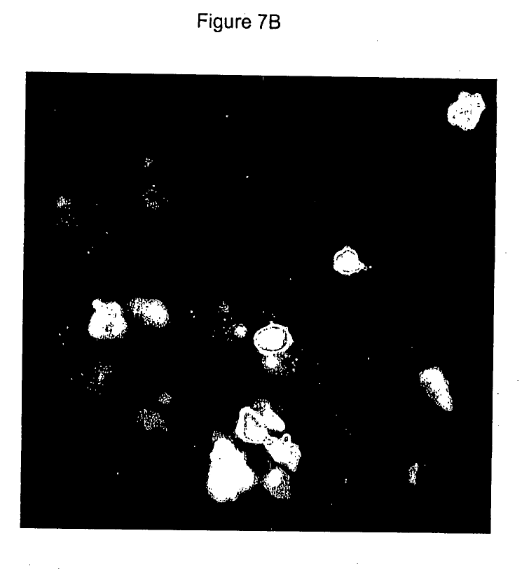

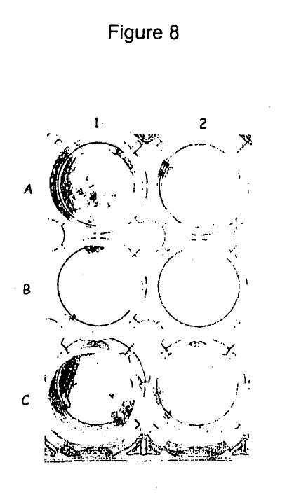

FIG. 8 Shows a photograph of CHO EM9 cells grown in the presence or absence of methanesulfonic acid ethyl ester (EMS), with or without Xenopus egg extract and with different levels of saponin;

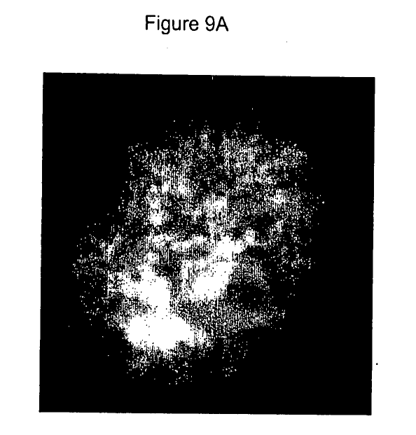

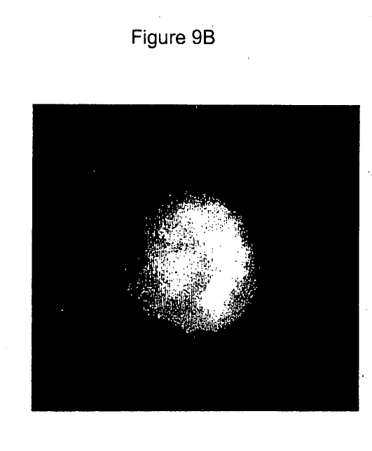

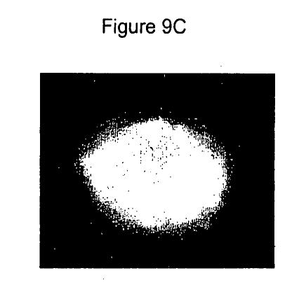

FIG. 9A Shows a micrograph of a human dermal fibroblast (HDF) permeabilised with digitonin and fixed immediately after treatment with Xenopus egg extract;

FIG. 9B Shows a micrograph of an HDF permeabilised with digitonin and fixed 2 h after treatment with Xenopus egg extract;

FIG. 9C Shows a micrograph of a further example of an HDF treated as described in FIG. 9B;

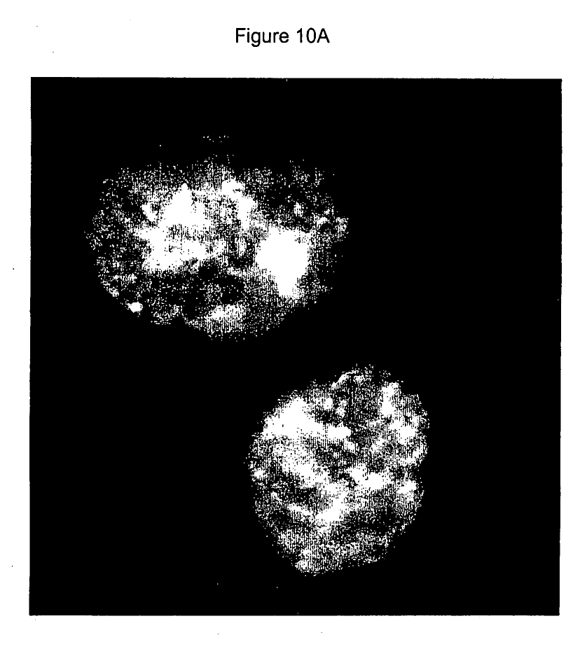

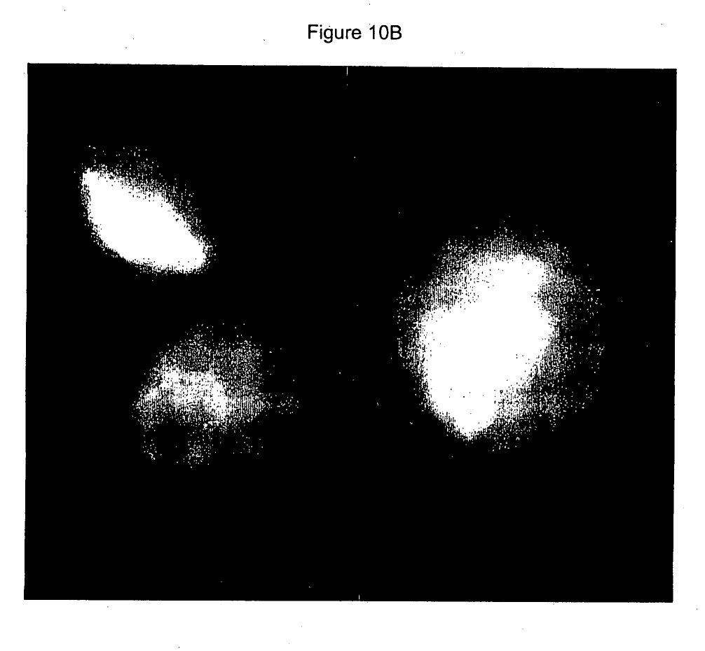

FIG. 10A Shows a micrograph of HDF cells permeabilised with digitonin and treated with EC P19 cell extract in the absence of an Energy Regeneration Mix for 2 h; and

FIG. 10B Shows a micrograph of HDF cells permeabilised with digitonin and treated with EC P19 cell extract in the presence of Energy Regeneration Mix for 2 h.

### EXPERIMENTAL

We now describe various methods used in the invention to acquire high efficiency reprogramming of target somatic cells. Reprogramming of somatic cells usually involves the following steps:

1) Preparation of concentrated extracts from whole, part, or derivative of the reprogramming cells, from cultures growing asynchronously or from cultures synchronised at a single point in the cell cycle;

2) Permeabilisation of the outer cell membrane of the target somatic cells and application of the reprogramming extract to the permeabilised cells or, alternatively, microinjection of the cellular extract, in order to deliver the reprogramming factors to the cell and, ultimately, the nucleus of the target cell;

3) Monitoring of the reprogramming event; and

4) Repair of the cellular membrane of the target cell.

Cell Culture

In all of these experiments cells were grown in standard cell culture media including but not restricted to Dulbecco's modified Eagle's medium (DMEM) or α-minimal essential medium (αMEM) in a humidified incubator at 37° C. and 5-10% CO₂. Media were supplemented, except where indicated, with 10% fetal calf serum (FCS) and 2 mM L-glutamine. Adherent cultures were grown to subconfluence (50-75%).

1.1) Preparation of Concentrated Extracts

In the preparation of extracts, the following chemicals were or may be added for the purposes described:

i) Phenylmethylsulfonyl fluoride (PMSF), chymostatin, leupeptin, aprotinin, antipain, pepstatin A, in order to prevent protein degradation by cellular proteases;

ii) Dithiothreitol (DTT) in order to prevent protein inactivation through oxidation;

iii) Cytochalasins B or D in order to inhibit intermediate filament formation and stability and thereby aid release of the mitotic/meiotic spindle or nucleus from the cell;

iv) β-glycerophosphate or vanadate in order to inhibit protein dephosphorylases (phosphatases) that may inhibit protein function by removing essential phosphate groups;

v) Energy Regeneration System/Mix that may include creatine phosphate, creatine kinase, ATP, GTP, MgCl₂, that supplies biochemical energy to the extract-based in vitro experiments;

vi) Glycerol and/or sucrose in order to stabilise protein extracts, during their preparation and/or storage; and/or

vii) RNAse inhibitors in order to prevent RNA degradation by cellular RNA-degrading enzymes.

In general, the cellular extracts described were prepared in the absence of added detergents in order to prevent subsequent inhibition of biochemical reactions.

Materials and Methods

Preparation of Cytosolic and Mitotic Extracts

Cytosolic extracts were made from cultures of human Embryonal Carcinoma (EC) cell lines, 2102E, Ntera2 (NT2) or murine EC cell lines, P19, F9, or PCC4, growing asynchronously or from cultures synchronised in in the cell cycle at two points in G1 or in mitosis. In order to synchronise cells in mitosis, DMEM medium was supplemented with 10 ng/ml Nocodazole and the culture was incubated in a humidified incubator at 37° C. and 5% CO₂ for 24-48 hours. (Alternatively, cells could be synchronised in mitosis by incubation in the presence of Vincristine, Colchicine, or Taxol.) Extracts could also be prepared from cells synchronised at two points in G1. In order to synchronise cells in G1 at the restriction point (Pardee, 1974, Proc. Natl. Acad. Sci. USA 71: 1286-1290), the growth medium was removed from the cells, the cells were washed in DMEM alone to remove residual FCS and the cells were then incubated for 48-72 hours in low-serum conditions (DMEM supplemented with 0.1% FCS and 2 mM L-glutamine). Cells were also synchronised at the G1-S phase boundary by replacing low serum medium after 48-72 hours, with DMEM supplemented with 10% FCS, 2 mM L-glutamine, and 300 μM L-Mimosine, and incubating the culture for a further 12-24 hours.

Asynchronous and synchronised cells were collected by centrifugation, the cell pellets were washed twice in 5-10 volumes ice-cold Phosphate Buffered Saline (PBS), followed by resuspension of the cell pellet in ice-cold Cell Extract Buffer [CEB; 50 mM PIPES (pH 7.4), 50 mM KCl, 50 mM EGTA, 2 mM MgCl₂, 1 mM DTT, 10 μM cytochalasin B, 1 mM PMSF]. All subsequent steps were carried out at 0-4° C. The cells were allowed to swell on ice for 10-15 minutes. The cells were then centrifuged and resuspended in an equal volume of CEB, centrifuged to pellet once more, and excess CEB was withdrawn from the pellet in order to prevent dilution of the final cellular extract. The cell pellet was snap-frozen in liquid nitrogen and stored at −80° C. The frozen pellet was thawed quickly at 37° C., and an additional 1 mM PMSF added. Alternatively, the cell pellet could be homogenised immediately after the final CEB wash. The cell pellet was transferred to a 2 mL Dounce homogeniser and the cells lysed by gentle homogenisation with 10-20 strokes of the Dounce using a tight-fitting pestle. Cell lysis was monitored by microscopic examination of an aliquot of the homogenate. Cell debris, including disrupted cellular membranes and intact nuclei, was removed by centrifugation at 4° C. for 10-15 minutes at 20,000-100,000×g. The final extract was snap-frozen and stored under liquid nitrogen in small aliquots (25-100 μl). The concentration of extracts prepared in this way was typically 5-50 mg protein/ml.

Alternatively, cells were resuspended after collection and washing with PBS, in 2.5 volumes of EBS Buffer [80 mM β-glycerophosphate, 20 mM EGTA, 0.1 M Sucrose, 15 mM MgCl₂, 1 mM DTT, 2 mM ATP] and allowed to swell on ice for 5-15 minutes. The cells were centrifuged to pellet and the pellet resuspended in 0.8 volumes EBS Buffer containing 1 mM PMSF. The cells were disrupted as described (above) and the homogenate centrifuged (435,000×g, 5 minutes, 4-10° C.). The resulting supernatant was centrifuged again as previously for 30 minutes. Extract prepared in this way was snap-frozen in small aliquots (50-500 μl) and stored at −80° C. The extract was desalted before use as described (below).

Preparation of Nuclear and Cytoplasmic Extracts

Cells were collected and washed as described for cytoplasmic extracts (above) with the exception that Buffer A [10 mM HEPES, (pH 7.9), 1.5 mM MgCl₂, 10 mM KCl, 0.5 mM DTT, 0.5 mM PMSF] was substituted for CEB. Additionally, 25% sucrose was added to some preparations in order to stabilise mitochondria and proteins. The extracts were prepared according to Dignam et al. (1983, Methods Enzymol. 101: 582-598) and Dignam (1990, Methods Enzymol. 182: 194-203). The cells were allowed to swell on ice and then homogenised as described above. Nuclei and debris were collected by centrifugation (1000×g, 10 minutes, 4-10° C.). The cytosolic fraction was removed and to it was added 0.11 volumes Buffer B [0.3 mM HEPES, (pH 7.9),1.4 M KCl, 30 mM MgCl₂], and this was stirred in an ice-water bath for a minimum of 30 minutes. The cytosolic fraction was then further centrifuged (100,000×g, 60 minutes, 4-10° C.) then optionally dialysed against 20 volumes of Buffer C [20 mM HEPES, (pH 7.9), 25% glycerol, 0.42 M KCl, 1.5 mM MgCl₂, 0.2 mM EDTA, 0.5 mM DTT, 0.5 mM PMSF] or Buffer C′ [as Buffer C but 0.6 M KCl]. Finally, particulate matter was optionally removed by further centrifugation (25,000×g, 20 minutes, 4-10° C.).

The nuclear/debris fraction from the first centrifugation step was further centrifuged (25,000×g, 20 minutes, 4-10° C.) and all remaining Buffer A was removed from the resulting pellet. The pellet was resuspended in Buffer C or C′ at a concentration of 2.5 ml/10⁹ cells. If necessary, the pellet was homogenised by 10-20 strokes of a Dounce homogeniser with tight-fitting pestle to disrupt the nuclear envelope. The nuclear lysate was then stirred gently on an ice-water bath for a minimum of 30 minutes. The nuclear lysate was then centrifuged (25,000×g, 30 minutes, 4-10° C.) then optionally dialysed against 50 volumes of Buffer D [20 mM HEPES, (pH 7.9), 20% glycerol, 100 mM KCl, 0.2 mM EDTA, 0.5 mM DTT, 0.5 mM PMSF]. Finally, particulate matter was optionally removed by further centrifugation (25,000×g, 20 minutes, 4-10° C.).

Optionally instead of dialysis, the cytosolic and nuclear fractions were passed over a desalting columns and the fractions collected by elution through Buffer D or EBS Buffer in which latter case the fractions were supplemented with Buffer A′ [20 mM β-glycerophosphate, 25 mM EGTA, 10 mM MgOAc, 50 mM KOAc].

The resulting cytoplasmic and nuclear extracts were snap frozen and stored under liquid nitrogen in small aliquots. The presence of sucrose and/or glycerol in the final extracts served to stabilise the proteins for long-term storage.

Preparation of Whole Cell Extracts

Cells were collected by centrifugation and washed in PBS as described above except that cells were washed and resuspended in lysis buffer [20 mM HEPES, (pH 8.2), SmM MgCl₂, 10 mM EDTA, 1 mM DTT, 20 μg/ml cytochalasin B, 0.5 mM PMSF and 10 μg/ml each of following protease inhibitors: cytochalasin, leupeptin, aprotinin and pepstatin]. The liquid was removed from the final cell pellet and this was snap-frozen in liquid nitrogen. The frozen pellet was quickly thawed by immersion in a 37° C. water bath. The cells, including nuclei, were disrupted by sonication with a tip sonicator (2×2 minutes) immersed in the cell mixture. Cell debris was cleared from the homogenate by centrifugation (14,000×g, 15 minutes 4-10° C.). The extract was snap-frozen and stored in small aliquots under liquid nitrogen.

Preparation of Extracts From Frog Eggs or Oocytes

Extracts were made from the eggs or oocytes of Xenopus laevis (Miake-Lye & Kirschner, 1985, Cell 41: 165-175; Murray & Kirschner, 1989, Nature 339: 275-280; Holloway et al., 1993, Cell 73: 1393-1402). The eggs were collected, washed in water, transferred to MMR Buffer [0.1 M NaCl, 2 mM KCl, 1 mM MgCl₂, 2 mM CaCl₂, 5 mM HEPES, 0.1 mM EDTA; pH 7.8], and washed several times in MMR. The eggs were dejellied using 2% cysteine (pH 7.8) washes over 5-10 minutes. The dejellied eggs were washed with MMR. The eggs were transferred into X3 [100 mM KCl, 1 mM MgCl₂, 0.1 mM CaCl₂, 10 mM HEPES, 50 mM sucrose; pH 7.7] and washed 4 times with XB, then twice with XB containing 1 μg/ml protease inhibitors (leupeptin, pepstatin, chymostatin), 5 mM EGTA, and an additional 1 mM MgCl₂. The eggs were transferred to an appropriate centrifuge tube in 1 mL XB/EGTA/protease inhibitors with added 10 μg/ml cytochalasin D. The eggs were centrifuged (1,000 rpm, 2 minutes, ambient temperature) in order to exclude as much liquid as possible in order that the final extract not be diluted. The eggs were then subjected to a crushing centrifugation (for example 10,000 rpm, 10 minutes, 16° C.). The golden-coloured cytoplasm was removed and 10 μg/ml protease inhibitors, 1 μg/ml cytochalasin D, and 1/20 of the volume of Energy Regeneration Mix (150 mM Creatine phosphate, 20 mM ATP, 20 mM MgCl₂, 2 mM EGTA) added. The extract was clarified by centrifugation (for example 14,000 rpm, 10 minutes, 4-10° C.). Sucrose to 150 mM was added before snap-freezing and storing in small aliquots (25-100 μl) under liquid nitrogen.

Extracts were prepared from oocytes in exactly this manner with the exceptions that the oocytes did not require removal of the jelly coat and the crushing centrifugation was performed at higher speed (for example, 20, 000 rpm).

Results

FIG. 1A. Asynchronous 2102E cells prior to homogenisation. Cells collected and washed as described were resuspended in CEB. A small aliquot of cells was removed and placed on a glass microscope slide for observation. As can be seen in the example illustrated in this figure, intact cells had rounded morphology with a discrete cell boundary, and some degree of internal cell structure was discernible. The surrounding medium is light in appearance and contained a minimum of particulate matter. Magnification is 400×.

FIG. 1B. Asynchronous 2102E cells after 20× Dounce homopenisation. Cells were collected as described and subjected to gentle lysis by Dounce homogenisation (20×). A small aliquot of the homogenate was removed for microscopic examination. In the example illustrated in the figure, it can be seen that cells were no longer intact and had lost their discrete cell boundary and any discernible internal cell structure. The disrupted cell debris would have included nuclei released from homogenised cells. In addition, the surrounding medium can be seen to contain particulate matter indicating the release of cellular contents into the medium after lysis by homogenisation. Magnification is 400×

1.2) Synchronisation of EC Cells in Mitosis

Materials and Methods

Adherent cultures of human Embryonal Carcinoma (EC) cell line, 2102E, were grown to subconfluence (50-75%) in DMEM complete [Dulbecco's modified Eagle's medium (DMEM) supplemented with 10% fetal calf serum (FCS) and 2 mM L-glutamine] in a humidified incubator at 37° C. and 5% CO₂. In order to synchronise cells in mitosis, DMEM complete was supplemented with 0-40 ng/ml Nocodazole and the culture was incubated in a humidified incubator at 37° C. and 5% CO₂ for 24 hours.

Cultures treated with 0, 10, 20, and 40 ng/ml Nocodazole were washed free of medium with PBS and fixed in ice-cold 70% ethanol in PBS, on ice or at 4° C. for 30 minutes or overnight. The ethanol was washed away with two rinses of PBS. The last wash was withdrawn and replaced with staining solution (5 volumes of PBS, 4 volumes of glycerol, 1 volume of water) including 1 μg/ml of the DNA-intercalating dye, Hoechst 33342. After 5 minutes in staining solution, the cells were observed by microscopy.

Synchronisation of EC cells in Mitosis (UV)

FIG. 2A. Asynchronous 2102E Cells

EC 2102E cells were treated with 0 ng/ml Nocodazole, fixed and stained as described above. The cells were examined under ultraviolet illumination. Since chromosomes become tightly condensed during mitosis, cells in mitosis stain brightly with Hoechst 33342. The figure illustrates an example of an asynchronous population of 2102E cells containing very few bright-staining cells indicating that the majority of cells were not in mitosis. Those cells that appear bright staining represent the proportion of cells that may normally be found in mitosis in an asynchronous culture. Magnification is 400×.

FIG. 2B. 2102E Cells Treated With Nocodazole

EC 2102E cells were treated for 24 hours with 10-40 ng/ml Nocodazole, fixed and stained as described above. The cells were examined under ultraviolet illumination. The figure shows an example of a culture treated with 10 ng/ml Nocodazole for 24 hours. The population contained an increased proportion of bright-staining cells relative to the control asynchronous culture illustrated in the previous figure. Cells treated with 15-40 ng/ml Nbcodazole contained a similarly higher proportion of bright-staining cells however this correlated with an increased proportion of dead/dying cells as the concentration of Nocodazole was increased. Magnification is 400×.

Synchronisation of EC Cells in Mitosis (Transmitted Light)

FIG. 3A. Asynchronous 2102E Cells

The figure illustrates an example of an asynchronously growing culture of 2102E cells (Magnification 400×) with features characteristic of EC cells: adherent aspect, growth in tight clusters within uniform borders, prominent nucleoli, and large nucleus/cytoplasm ratio.

FIG. 3B. 2102E Cells Treated With Nocodazole

The figure illustrates an example of 2102E EC cells after treatment for 24 hours with 10 ng/ml Nocodazole. As can be seen in the figure, relative to untreated controls, the Nocodazole-treated cultures contain a proportion of cells that have lost their adeherent aspect and “rounded up”, a feature characteristic of cells in the mitotic phase of the cell cycle.

Discussion

Since the nuclei were discarded in the centrifugation step to remove cell debris, extracts made using the methods described above from asynchronously growing cells and cells synchronised in G1 yielded soluble cytosolic components excluding nuclear components. However, since the nuclear membrane breaks down at mitosis or meiosis, in the case of germ cells, thus releasing these components into the cytosol surrounding the nucleus, extracts prepared from cells synchronised in mitosis/meiosis included cytosolic components and components normally found in the nucleus. Similarly, in eggs or oocytes the nucleus ceases to exist as a discrete organelle, the nuclear membrane having been disassembled in the process of entering meiosis. The inclusion of nuclear components in the cellular extract is an important feature of the invention since it would appear that remodelling of the chromatin that accompanies reprogramming requires specific factors normally resident in the nucleus (Kikyo et al., 2000, supra).

Extracts made from cells isolated from a particular phase of the cell cycle may contain factors effective in deprogramming that are preferentially present and active only during that particular phase. Cells treated with Nocodazole (Vincristine, colchicines, taxol) collect in mitosis. Eggs and oocytes are arrested in their development in metaphase I or II, respectively, of meiosis. Cells collected from a single cell cycle phase can be expected to yield the maximal concentration of the particular factors active within that phase. In addition, during M-phase (mitosis or meiosis) the nuclear envelope is broken down and nuclear and cytoplasm components can be found within the same soluble cytosol at relative physiological concentrations and stoichiometry.

2.1) Permeabilisation/Microinjection of and Delivery to Target Somatic Cells

Materials and Methods

Permeabilisation with Saponin

Adherent cultures of subconfluent Chinese Hamster Ovary (CHO) EM9 cells were washed free of growth medium with PBS. The cultures were treated with 0-50 μg/mL Saponin, a non-ionic detergent, in physiological buffer (PB; Jackson et al., 1988, J. Cell Sci. 365: 378-390) [100 mM potassium acetate, 30 mM KCl, 10 mM Na₂HPO₄, 1 mM MgCl₂, 1 mM disodium ATP, 1 mM DTT, 0.2 mM PMSF, pH 7.4]. The cells were treated by applying the saponin for 1-2 minutes, immediately removing the detergent solution, washing twice with PBS, and twice with αMEM, and finally applying growth medium.

Cell permeability was monitored immediately after saponin treatment and again 24 hours later. A 2% solution of Trypan Blue was mixed with an equal volume of PBS. Medium was washed from the cell cultures and the I % Trypan Blue solution was applied. Cells were viewed immediately by light microscopy.

Permeabilisation with Streptolysin O

Streptolysin O (SLO) is a bacterial toxin purified from Streptococcus pyogenes, that permeabilises the outer cellular membrane and permits uptake of large or charged molecules, including proteins (Walev et al., 2001, Proc. Natl Acad. Sci. USA 98: 3185-3190) into the cell cytoplasm. The pores formed can be resealed by addition of FCS or calcium to the incubation media. CHO EM9 were washed with PBS and the cells were permeabilised with 5 to 20 units/10⁶ cells of activated streptolysin O in serum-free medium for 10 minutes at 37° C. To reseal plasma membranes, 10% serum containing media was added and cells were incubated for a further 30 minutes at 37° C. and 5% CO₂.

Using this procedure (Giles et al., 1998, Nucleic Acids Res. 26: 1567-1575) some cells were permeabilised reversibly, some cells were irreversibly permeabilized (i.e. killed), while others remained unpermeabilized. Illustrative examples are detailed. To assess the permeabilisation efficiency cells were stained with 10 μM fluorescein diacetate (FDA), a marker for viable cells, which was added during incubation in serum free medium, and 10 μg/ml of propidium iodide (PI), a marker for dead cells, which was added after plasma membranes had been resealed. Cells were analysed by flow cytometry: green only cells were permeabilised and resealed, red only or red and green cells were dead, while colourless cells were non permeabilised.

Permeabilisation With Digitonin

The outer cellular membrane of target cells can also be permeabilised using digitonin according to (Wilson et al., 1995, Biochem. J. 307: 679-687). Cells are washed with PBS and released from the growing surface using Trypsin-EDTA then centrifuged to pellet. The cell pellet is washed in KHM buffer [110 mM KOAc, 2 mM MgOAC, 20 mM HEPES (pH 7.2)] The pellet is resuspended in ice-cold KHM buffer to which digitonin is added to a final concentration of 40 μg/ml and incubated on ice for 5 minutes. Enough ice-cold KHM buffer is then added to double the volume and the whole centrifuged to pellet. The liquid is removed and the cells are resuspended in ice-cold HEPES buffer [KOAc 50 mM, HEPES 90 mM (pH 7.2)] then place on ice for 10 minutes. Permeabilisation of the cells can be monitored by staining with trypan blue as described (above).

Microinjection

Cultures of the human osteosarcoma cell line 143B were plated 24 hours prior to microinjection in order that the cells be well adhered and spread on the growing surface and at a concentration such that cells were well separated from each other.

An aliquot of mixed nuclear and cytosolic cell extracts, prepared as described above from NTERA2 D1 cells, was thawed and fluorescein isothiocyanate (FITC)-conjugated-dextran with a molecular weight of 70,000 kilodaltons added as a marker of successful microinjection. The mixture of extract and marker was centrifuged at 10,000×g for 15 minutes in order to pellet particulate and aggregated matter and the cleared supernatant was removed to a fresh tube. The extract was loaded into a microinjection needle with 0.5 μm inner diameter and outer diameter of 0.5-1.1 μm. The microinjection needle was mounted onto a microinjection apparatus with micromanipulator and adjusted so as to come into contact with a target cell. The cell membrane was punctured with the needle and the extract delivered (0.1-40 μl) to the cell. Alternatively, the extract was delivered directly to the nucleus. The cells were examined directly for the presence of FITC-dextran within the cell.

Results

Saponin 0 Hours

FIG. 4A. Mock-treated Cells

Cells were treated as described with the exception that no saponin was added to the physiological buffer. The cells were then stained with trypan blue. Cells that have had their outer cell membrane permeabilised will readily take up trypan blue which stains the cytoplasm of the cell dark blue. As the example in the figure illustrates, cells treated with PB alone did not take up trypan blue indicating that the cell membrane was intact.

FIG. 4B. 30 μg/ml Saponin

An example of EM9 cells treated with this concentration of saponin are illustrated. As can be seen in the figure, at this concentration of saponin approximately 50% of the cells were permeabilised and stained darkely with the application of trypan blue.

FIG. 4C. 50 μg/ml Saponin

As can be seen in the figure, at this concentration of saponin approximately 80% of the cells were permeabilised and stained darkly with the application of trypan blue.

Saponin 24 Hours

FIG. 5A. Mock-treated Cells

24 hours after cells were mock-treated the cells remained impervious to trypan blue and no dark-staining cells can be seen in the example illustrated.

FIG. 5B. 30 μg/ml Saponin

As with the mock-treated cells, 24 hours after treatment with 30 μg/ml saponin, there were no dark-staining cells apparent in this example illustration.

FIG. 5C. 50 μg/ml Saponin

In contrast as can be seen in the example, at this concentration of saponin approximately 30% of the cells stained darkly with the application of trypan blue. The positive trypan blue staining indicates that the outer membrane of these cells was irreversibly permeabilised and further indicates that these cells were dead.

Streptolysin O

FIG. 6A. Mock Treatment

CHO EM9 cells were treated exactly as described with the exception that SLO was excluded from the treatment. Cells were stained FDA and PI exactly as described and analysed by flow cytometry. The figure shows a plot of the flow cytometric analysis for this sample, divided into four quadrants. Each quadrant is labelled at its corner with the percentage of cells in the sample that fall within that particular quadrant. The bottom left quadrant contains cells that were unpermeabilized and thus remained largely unstained, the upper left quadrant contains cells that stained positively with PI (red), the bottom right quadrant contains cells that stained positively for FDA (green). The upper right quadrant would contain cells stained with both FDA and PI (red and green).

Mock treatment resulted in 5.3% cells being irreversibly permeabilised. This represents the background of dead cells within the population. A further 1% of cells took up FDA without SLO permeabilisation.

FIG. 6B. 5 Units SLO

Similar to mock treated controls, approximately 5% of cells within the population stained with PI suggesting that this concentration of SLO did not increase the number of dead cells. However, this concentration failed to increase the ingress of FDA into cells relative to the mock-treated control indicating that cells remained unpermeabilised.

FIG. 6C. 10 Units SLO

At this concentration of SLO, 7.6% of cells became irreversibly permeabilised and 6.1% of cells became reversibly permeabilised as indicated by uptake of FDA but not of PI. This represents an increase in permeabilised cells of approximately 5% over mock treated cells.

FIG. 6D. 20 Units SLO

At this concentration of SLO, 9% of cells were irreversibly permeabilised. The percentage of reversibly permeabilised cells was similar to mock-treated control.

Microinjection

FIG. 7A. Microinjected and Uninjected Cells (White and UV Light)

In this example some 143B cells were injected with extract containing FITC-dextran. The cells were visualised by microscopy (400×) with illumination by ultraviolet and transmitted white light. The injected cells are visibly fluorescent in this image due to the FITC-dextran, whilst the uninjected cells remain darker in aspect and non-fluorescent.

FIG. 7B. Microinjected and Uninjected Cells (UV Light)

The same cells shown in Part 1 were subjected to UV light alone so that only the microinjected cells were visible due to their fluorescent labelling with FITC-dextran

Discussion

The specific examples described demonstrate that target cells can be reversibly permeabilised. Subsequently, solutions in which marker molecules trypan blue, FDA, PI, or FITC-dextran have been included allows visualisation of ingress of molecules through the permeabilised cell membrane into the cytosol. That this is a reversible process is demonstrated by the survival as measured by exclusion of trypan blue 24 hours after treatment, of the majority of cells treated with 30 μg/ml saponin and by the staining with FDA but not PI of cells treated with 10 units/ml streptolysin O. These specific examples are germane to the invention since it is an object of the invention to allow ingress of molecules, preferably reprogramming molecules, into permeabilised cells. It is a further object of the invention that the cells so treated repair the permeabilised cellular membrane such that cells survive and furthermore proliferate in tissue culture conditions.

Alternatively and additionally, cells may be microinjected with protein extracts directly. The specific example demonstrates that a protein extract, including cytoplasmic and nuclear components, containing the marker molecule FITC-dextran was injected into the target cells. The specific example is germane to the invention since it is an object of the invention to microinject molecules, preferably reprogramming molecules, of significant size into target cells. The specific example demonstrates that molecules of at least molecular weight 70,000 kilodaltons can be introduced into the target cell by microinjection.

2.2) Delivery of a Functional Nuclear Protein

The CHO cell line EM9, is exquisitely sensitive to certain DNA damaging agents, including the alkylating agent methanesulfonic acid ethyl ester (EMS; Thompson et al., 1982, Mol. Cell Biol. 10: 6160-6171), and is unable to repair the lesions caused to the DNA by application of this agent. The failure of EM9 to repair EMS-induced DNA lesions is due to mutation in a single gene, XRCC1 (Thompson et al., 1990, Mol. Cell Biol. 10: 6160-6171). However, this defect can be alleviated by delivery of recombinant XRCC1 by electroporation to EM9 cells subsequent to the cells being exposed to EMS (Caldecott & Thompson, 1994, Ann. N.Y. Acad. Sci. 726: 336-339). XRCC1 is a highly conserved protein and has been shown to be present in groups as divergent as boney fish and human. In the specific example, extract prepared from Xenopus laevis eggs as described (above) was delivered to permeabilised CHO EM9 cells subsequent to EMS-induced DNA damage.

Materials and Methods

CHO EM9 (2.5×10⁴ cells/treatment) cells were exposed to methanesulfonic acid ethyl ester (1 mg/ml, 60 minutes, 37° C.) supplied in αMEM complete medium or left untreated as mock controls. After treatment, cells were washed free of EMS with two changes of PB. Cells were then permeabilised with 0, 30, or 60 μg/ml saponin as described (above). The cells were washed free of saponin as described (above) and incubated (37° C., 30 minutes) in the presence of Xenopus egg extract (for example 15 μl) or mock-treated with PB alone. αMEM complete medium was reintroduced to the cells and they were left to grow for two weeks. After two weeks the growth medium was removed, the residual medium washed away with PBS, and the cells were fixed with 70% ethanol. The fixed cells were then washed in PBS and stained with a solution of 1% methylene blue in 70% methanol.

Results

FIG. 8. The example illustrates an experiment demonstrating delivery of fully functional protein(s) to the cell nucleus. CHO EM9 cells would die without application of XRCC1 protein or a surrogate activity that can repair the DNA lesions caused by treatment with EMS.

In the figure the following samples are illustrated:

1) A1 30 μg/ml saponin, 1 mg/ml EMS, 15 μl extract;

2) B1 30 μg/ml saponin, 1 mg/ml EMS, 15 μl PB;

3) C1 30 μg/ml saponin, 15 μl PB;

4) A2 60 μg/ml saponin, 1 mg/ml EMS, 15 μl extract;

5) B2 60 μg/ml saponin, 1 mg/ml EMS, 15 μl PB;

6) C2 60 μg/ml saponin, 15 μl PB.

Comparing column 1 samples with column 2 samples illustrates that 60 μg/ml saponin treatment killed most cells while relatively more cells survive 30 μg/ml saponin treatment. Comparing sample B1 to sample C1, and to a lesser degree comparing B2 to C2, illustrates that (in the absence of treatment with extract) EMS treatment is lethal in EM9 cells. Comparing sample A1 to sample B1, and to a lesser degree A2 to B2, illustrates that application of extract to cells that had been treated with EMS allowed more EM9 cells to survive and proliferate relative to cells that had no extract applied. Finally, comparing samples A1 and C1 illustrates that addition of extract resulted in relatively more cells surviving saponin, since fewer cells survived in C1 than in A1 and lethality in C1 relative to A1 was solely due to saponin since C1 was not treated with EMS.

Discussion

One of the necessary steps of the invention is delivery to the target cell nucleus of proteins capable of affecting DNA structures. EMS treatment causes DNA lesions and chromosomal DNA resides in the nucleus. In EM9 cells, repair of the chromosomal DNA lesions caused by EMS was necessary for cell survival. That factors capable of effecting repair of these lesions gained access to the nucleus via the permeabilised outer cellular membrane was evidenced by the increased survival of cells that were treated with egg extract but not of cells that were mock-treated. This experiment illustrates that EM9 cells treated with EMS were “reprogramned” by application of a protein extract containing a specific factor essential for allowing repair of DNA damage. In order to allow repair of the damaged chromosomal DNA, necessarily, such a factor gained access to the nucleus.

In addition this example illustrates repair of the outer cellular membrane after permeabilisation. Comparing samples in which cells were treated with 30 μg/ml saponin but not with EMS or extract to samples treated with EMS and extract, it was clear that benefit was gained by the application of extract since even in the absence of EMS treatment, fewer cells survived without extract than survived with extract even though these latter cells were additionally treated with EMS.

3) Monitoring the Reprogramming Event

Nuclear Remodelling

It has been shown that isolated quiescent nuclei of Xenopus erythrocytes can be reactivated usingXenopus egg extract to provide isolated replicative nuclei (Wangh et al., 1995, supra). These nuclei did not constitute cells as such, as they had no cytoplasm bounded by an outer cell membrane: such experiments provide an example of a cell-free model or surrogate of nuclear reactivation. Similarly, Kikyo et al. (2000, supra) demonstrated in a cell-free model system the morphological remodelling of somatic nuclei incubated in soluble extracts made from Xenopus laevis eggs. Remodelling in this cell-free surrogate system was accompanied by loss of specific proteins from the chromatin of the nucleus and gain of other factors by ingress to the nucleus from the Xenopus egg extract. The surrogate system also demonstrated that remodelling was accompanied by changes in the morphological characteristics of the nuclei in remodelling extracts such that to a large extent nucleoli disappeared and the nucleus appeared smoother in aspect with less “granular” features.

In the specific examplesXenopus egg extracts or ECP19 cell extracts were applied to permeabilised human dermal fibroblasts (HDFs) in a remodelling surrogate assay.

Materials and Methods

HDFs were collected by centrifugation, washed once with PBS and once in Nuclear Preparation Buffer [NPB; 250 mM sucrose, 15 mM HEPES, (pH 7.7), 1 mM EDTA, 0.5 mM spermidine, 0.2 mM spermine, 1 mM DTT]. Protease inhibitors (10 μg/ml leupeptin, pepstatin A) were added, the pellet resuspended in NPB containing 40 μg/ml digitonin, and the cells allowed to permeabilise on ice for 5-10 minutes. Cells were centrifuged to pellet, washed twice in NPB, and finally resuspended in NPB. Meanwhile, the extract prepared from Xenopus eggs or EC cells was incubated at ambient temperature for 30 minutes and Energy Regeneration Mix was added to some samples as appropriate. Permeabilised cells (1×10⁵) were resuspended in extract (25 μl) and incubated at 22-37° C. for 0-2hours. After appropriate incubation 100 μl NPB was added and the samples centrifuged to pellet (15,000×g, 10 minutes 4-10° C.), washed in NPB and the centrifugation step repeated. Finally, the cell pellet was resuspended in 50 μl NPB, and fixed in formalin/PBS at ambient temperature. The sample was then viewed by microscopy (1000×) after addition of Hoechst 33342, a DNA intercalating dye that stains only the nucleus.

Results

Xenopus Extract Remodelling of HDFs

FIG. 9A. 0 Hours (Mock Treatment)

HDFs treated as described (above) to permeabilise the outer cellular membrane were mixed with Xenopus egg extract and immediately cells were fixed and stained with Hoechst 33342, effectively allowing no remodelling to occur. Although they did not appear to have prominent nucleoli, the example illustrative nuclei stained heterogeneously resulting in a “granular” or “speckled” appearance in the absence of remodelling extract.

FIG. 9B,C. 2 Hours

Permeabilised HDFs were incubated in Xenopus egg extract for 2 hours. In two illustrative examples, compared to mock-treated controls not incubated in extract, these nuclei stained homogenously, bad lost their “granular” or “speckled” appearance and now appeared “smooth” in aspect. In addition, the nuclei appeared smaller than those that had not been incubated in extract.

EC P19 Extract Remodelling of HDFs

FIG. 10A. 2 Hours−ERM (Mock Treatment)

Permeabilised HDFs were incubated for 2 hours in P19 extracts (nuclear and cytoplasmic prepared as described mixed in 1:1 ratio) in the absence of the Energy Regeneration Mix. Similar to the observations for the mock-control described for Xenopus egg extracts, the example illustrated demonstrates that nuclei were granular/speckled in aspect, heterogeneously staining, and effectively un-remodelled in appearance.

FIG. 10B. 2 Hours+ERM

Permeabilised HDFs incubated for 2hours in P19 extracts in the presence of ERM appeared, as in the example illustrated, extensively remodelled as evidenced by the loss of heterogeneous staining to take on a smooth, homogeneously-stained aspect.

Discussion

One aspect of the invention is that cells are reprogrammed via the infusion of extracts prepared from whole, part or derivative of the reprogramming cells. It is believed that chromatin is remodelled in order that reprogramming takes place. Treatment of permeabilised mammalian cells with cellular extracts to effect remodelling provided a surrogate of reprogramming by prepared cell extracts. Additionally, human dermal fibroblasts provide a feasible target for application of reprogramming in the development of treatment as outlined in the invention.

Although exchange of specific protein factors between Xenopus nuclei and Xenopus egg extracts has been observed in cell-free surrogate model systems by Kikyo et al. (2000, supra), and similar exchange of proteins is implied in experiments in which mammalian somatic nuclei have been introduced into enucleated mammalian oocytes to produce cloned animals, similar observations have not previously been noted in isolated somatic mammalian nuclei treated with extract preparations either from Xenopus eggs/oocytes or EC cells.

Assaying Specific Gene Expression

Successful reprogramming of target somatic cells by treatment with EC cell extracts or Xenopus egg extracts may alter specific gene expression in the target cells such that they would express one or more genes characteristic of de-differentiated or pluripotent cells. One method of determining that reprogramming has taken place is to assay specifically for expression of such a gene. In the specific example, cells that had been reprogrammed by application of EC cell or Xenopus egg extract would be expected to express markers of pluripotency since both extracts were prepared from pluripotent cells. Genes characteristically expressed in pluripotent cells include, but are not restricted to, Oct 3/4 and Sox2.

The stem cells produced by the methods described above may have pluripotent properties that closely resemble those of embryonic stem cells, so that the cells may be able to differentiate and initiate differentiation pathways that result in the formation of any cell type that may be found in the adult, embryo or in extra-embryonic tissues, given appropriate conditions. The reprogrammed cell will then express markers of differentiation that differ from the markers of differentiation that it originally expressed prior to reprogramming. For example, a thymocyte, once reprogrammed and allowed to differentiate anew, may express markers of endodermal differentiation including but not restricted to, laminin B1 (Chen & Gudas, 1996, J. Biol. Chem. 271: 14971-14980).

Material and Methods

In order to analyse gene expression an aliquot of cells or RNA (not exceeding 1 μl, equivalent to 10⁴-10⁶ cells or 10-1000 ng RNA) was subjected to reverse transcription and PCR amplification as described in Brady & Iscove (1993, Methods Enzymol. 225: 611-623) using the primer Not1dT (5′CAT CTC GAG CGG CCG CTT TTT TTT TTT TTT TTT TTT TTT T 3′; SEQ ID NO: 1) to produce polyA cDNA. The polyA cDNA was subjected to “TaqMan” real-time PCR using an ABI Prism 7700 System and Universal Master Mix (Applied Biosystems Inc.) with primers and probes (detailed below) designed using Primer Express (ABI) and according to the manufacturers instructions. Primers and probes were tested for their unique recognition of the desired gene/cDNA sequence using NCBI BLAST analyses.

In order to detect reaction products by TaqMan real-time PCR, probes were modified by inclusion of FAM and TAMRA fluorescent labels. FAM is tagged on the 5′ end and TAMRA on the 3′ end of the probe. The primers and probe anneal specifically to their target gene through sequence homology. Whilst the probe is bound to its homologous sequence, TAMRA quenches the fluorescent signal from FAM. During the PCR reaction, the probe is displaced from the DNA and FAM is cleaved from the probe. Having been displaced, FAM is no longer quenched by TAMRA, and FAM releases a fluorescent signal that is detected by the instrument. The amount of FAM cleaved from the probe during PCR, the amount of fluorescent signal, is directly proportional to the amount of starting template in the reaction.

The standard TaqMan reaction is 40 cycles as indicated in the manufacturer's instructions. The first cycle at which FAM can be detected by the instrument is called the “threshold” cycle (Ct) for the gene under investigation. If there were no template to amplify, no FAM would be cleaved from the probe (eg. no polyA cDNA was included in the reaction mix), and the Ct value would be 40 (i.e. fluorescence due to FAM cleavage was not detectable after 40 cycles). A reaction that produced no FAM signal upon completion of 40 cycles would be equivalent to a product in which no template for PCR had been included. A Ct value less than 40 indicates that the primers/probe recognised a target template and that FAM was cleaved as the PCR reaction displaced it from the template.

The primers and probes used for detection of murine and human Oct 3/4, Sox2, GAPDH, and laminin B1 are detailed below:

PCR Primers for Human and Mouse Genes

1. A method of producing a pluripotential mammalian stem cell (“stem cell”) from a target mammalian somatic cell (“target cell”) comprising:
(a) providing a medium comprising: a whole, partial, or derivative extract of a reprogramming cell, wherein the extract comprises soluble components of cytoplasm from the cell and nuclear factors, and wherein the extract is enriched for the nuclear factors;
(b) providing a target cell comprising a nucleus and an outer cellular membrane;
(c) introducing the medium into the target cell, wherein the medium causes reprogramming of the target cell nucleus to form a stem cell having a reprogrammed nucleus and an outer cell membrane from the target cell.

2. The method of claim 1, wherein the soluble components and/or the nuclear factors in the extract of the medium cause reprogramming of the target cell nucleus.

3. The method of claim 2, wherein the extract is from a reprogramming cell in a G1, G2, or M cell cycle phase.

4. The method of claim 2, wherein the extract is from a reprogramming cell in a metaphase to anaphase transition cell cycle phase.

5. The method of claim 4, wherein the cell cycle phase is induced by a synchronization agent.

6. The method of claim 5, wherein the synchronization agent is Nocodazole.

7. The method of claim 2, wherein the nuclear factors are obtained from a karyoplast isolated from the reprogramming cell.

8. The method of claim 2, wherein the nuclear factors are obtained from a nucleus isolated from the karyoplast or the reprogramming cell.

9. The method of claim 8, wherein the nuclear membrane of the reprogramming cell, the karyoplast, or the isolated nucleus is disrupted to release nuclear factors.

10. The method of claim 9, wherein the nuclear membrane is disrupted by sonication, isotonic bursting, and/or by using an homogenizer.

11. The method of claim 10, wherein the medium is introduced into the target cell following permeabilization of the outer cellular membrane of the target cell.

12. The method of claim 11, wherein permeabilization is achieved using a permeabilization agent.

13-14. (canceled).

15. The method of claim 11, wherein the permeabilization is achieved by using an electric pulse.

16. The method of claim 11, wherein the medium is injected into the target cell.

17. The method of claim 4, wherein the extract is from a reprogramming cell which has been pre-treated with an agent that causes enucleation of the cell.

18. The method of claim 17, wherein the agent is cytochalasin,.

19. The method of claim 17, wherein the extract is provided as enucleated whole cytoplasm.

20. The method of claim 17, wherein the extract is provided as a derivative of the cytoplasm of the reprogramming cell.

21. The method of claim 17, wherein the extract is provided as a derivative of an isolated karyoplast.

22. The method of claim 21, wherein the extract and/or medium is supplemented with a ribonuclease inhibitor and/or a proteinase inhibitor.

23. The method of claim 21, wherein the extract and/or medium is supplemented with an antioxidant.

24. The method of claim 21, wherein the extract and/or medium is supplemented with an agent which inhibits protein dephosphorylation.

25. The method of claim 21, wherein the extract and/or medium is supplemented with an energy regeneration mix comprising: creatine kinase, and/or creatine phosphate, and/or ATP, and or GTP, and/or MgCl₂.

26. The method of claim 21, wherein the extract and/or medium is supplemented with an agent that stabilizes the extract and/or medium.

27. The method of claim 1, further comprising:
(d) incubating the target cell in conditions conducive to the reconstruction and/or repair of the outer cellular membrane to form the stem cell.

28. The method of claim 20, wherein the reprogramming cell is a germ cell.

29. The method of claim 28, wherein the germ cell is an egg cell or an embryonal carcinoma cell.

30. The method of claim 28, wherein the reprogramming cell is a mammalian cell.

31. The method of claim 27, wherein the target cell is selected from the group comprising: a thymocyte, peripheral blood lymphocyte, epidermal cell, buccal cavity cell, cumulus cell, bone marrow stem cell, nervous system stem cell, or gut stem cell, or is obtained from established cell lines, tissues, or organs of an adult mammal.

32. The method of claim 31, wherein the target cell nucleus is encapsulated in a support medium.

33. The method of claim 32, wherein the support medium is agarose.

34. The method of claim 27, further comprising:
(e) isolating at least one stem cell.

35. The method of claim 34, further comprising:
(f) culturing the stem cell in conditions conducive to propagate the stem cell.

36. A method of producing a stem cells, comprising incubating more than one target cell in the medium of claim 33 to induce simultaneous reprogramming of target cell nuclei.

37. A stem cell obtained by the method of claim 36.

38. The stem cell of claim 37, wherein the stem cell has the ability to proliferate in culture in an undifferentiated state.

39. The stem cell of claim 38, wherein the stem cell has at least one pluripotential characteristic.

40. The stem cell of claim 39, wherein the stem cell has the ability to differentiate into one of at least two selected tissue types.

41. The stem cell of claim 40, wherein the stem cell expresses at least one selected marker.

42. The stem cell of claim 41, wherein the selected marker is selected from group consisting of one or more of the following: Oct3/4, Sox2, SSEA-1 (−), SSEA-3 (+), SSEA-4 (+), TRA-1-60 (+), TRA-1-81 (+), lacZ, and GFP.

43. The stem cell of claim 42, wherein the stem cell possesses telomerase activity.

44. The stem cell of claim 43, wherein the stem cell possesses a chromosomal methylation pattern characteristic of pluripotential cells.

45. The stem cell of claim 44, wherein the stem cell is human.

46. A cell culture comprising at least one stem cell produced by the method of claim 36.

47. A cell culture comprising at least one stem cell of claim 44.

48. The stem cell of claim 37, wherein the stem cell is produced in a tissue selected from the group consisting of one or more of the following: neural, smooth muscle, striated muscle, cardiac muscle, bone, cartilage, liver, kidney, respiratory epithelium, haematopoietic cells, spleen, skin, stomach, and intestine.

49. A tissue comprising at least one stem cell of claim 44, wherein the tissue is used in transplantation.

50. A therapeutic composition comprising at least one stem cell of claim 44.

51. The therapeutic composition of claim 50, further comprising a therapeutically acceptable excipient, diluent, or carrier.

52. The therapeutic composition of claim 51, wherein the therapeutic composition is used in tissue transplantation.

53. A method of inducing differentiation of at least one stem cell comprising:
(a) providing the stem cell of claim 44;
(b) culturing the stem cell under conditions which cause differentiation of the stem cell; and optionally
(c) storing the differentiated stem cell prior to use under suitable storage conditions.

54. A method of producing a tissue comprising:
(a) providing the stem cell of claim 44; and
(b) culturing the stem cell under conditions which cause proliferation of the cell to form a tissue.

55. A method of treating a condition or a disease requiring tissue transplantation comprising:
(a) providing a tissue produced by the method of claim 54;
(b) transplanting the tissue into a subject in need thereof; and
(c) treating the subject in need thereof under conditions which allow the acceptance of the transplanted tissue by the subject in need thereof.

56. A therapeutic composition comprising a tissue produced by the method of claim 54.

57. The therapeutic composition of claim 56, further comprising a therapeutically acceptable excipient, diluent, or carrier.

58. The stem cell of claim 37, wherein the stem cell is used for screening of compounds that have the potential to treat diseases.

59. A differentiated stem cell produced by the method of claim 53, wherein the differentiated stem cell is used for screening compounds that have the potential to treat diseases.

60. The stem cell of claim 37, wherein the stem cell is used to study organ development.

61. A reprogrammed mammalian cell produced by transfer of an extract that comprises components of a whole, part, or derivative of another type of cell, wherein the extract is enriched for nuclear factors.

62. The extract and/or medium of claim 26, wherein the extract and/or medium is obtained from a mammalian reprogramming cell.

63. The method of claim 12, wherein the permeabilization agent is selected from the group consisting of saponin, digitonin, and streptolysin O.

64. The method of claim 63, wherein the concentration of saponin is in the range of from about 5 μg/ml to about 45 μg/ml.

65. The method of claim 64, wherein the concentration of saponin is in the range of from about 10 μg/ml to about 35 μg/ml.

66. The method of claim 65, wherein the concentration of saponin is about 30 μg/ml.

67. The method of claim 63, wherein the concentration of streptolysin O is in the range of from about 1 unit/ml to about 20 units/ml.

68. The method of claim 67, wherein the concentration of streptolysin O is in the range of from about 5 units/ml to about 10 units/ml.

69. The method of claim 18, wherein the cytochalasin is cytochalasin B or cytochalasin D.

70. The method of claim 69, wherein the concentration of cytochalasin B or cytochalasin D is in the range of from about 1 μM to about 20 μM.

71. The method of claim 23, wherein the antioxidant is dithiothreitol and/or β-mercaptoethanol.

72. The method of claim 71, wherein the concentration of dithiotheitol is in the range of from about 0.5 mM to about 5 mM.

73. The method of claim 71, wherein the concentration of β-mercaptoethanol is in the range of from about 100 mM to about 500 mM.

74. The method of claim 24, wherein the agent is β-glycerophosphate and/or vanadate.

75. The method of claim 25, wherein the concentration of creatine kinase is in the range of from about 50 μg/ml to about 100 μg/ml.

76. The method of claim 25, wherein the concentration of creatine phosphate is in the range of from about 10 mM to about 20 mM.

77. The method of claim 25, wherein the concentration of ATP is in the range of from about 1 mM to about 2 mM.

78. The method of claim 25, wherein the concentration of GTP is in the range of from about 1 mM to about 2 mM.

79. The method of claim 25, wherein the concentration of MGCl₂ is about 1 mM.

80. The method of claim 26, wherein the agent is glycerol and/or sucrose.

81. The method of claim 80, wherein the concentration of sucrose is in the range of from about 5% v/v to about 50% v/v.

82. The method of claim 28, wherein the egg cell is a Xenopus laevis egg cell.

83. A tissue produced by the method of claim 54, wherein the tissue is used for screening compounds that have the potential to treat diseases.

84. A differentiated stem cell produced by the method of claim 53, wherein the differentiated cell is used to study organ development.
